(19) United States
(12) Patent Application Publication
(10) Pub. No.: US 2025/0261839 A1
(43) Pub. Date: Aug. 21, 2025

(54) MULTIMODAL CAPSULE-BASED LIGHT DELIVERY, COLLECTION, AND DETECTION SYSTEMS AND METHODS

(71) Applicant: The General Hospital Corporation, Boston, MA (US)

(72) Inventors: Andrew Thrapp, Boston, MA (US); Guillermo Tearney, Boston, MA (US); Evaggelia Gavgiotakis, Boston, MA (US)

(21) Appl. No.: 18/857,425

(22) PCT Filed: Apr. 18, 2023
(86) PCT No.: PCT/US2023/065883

§ 371 Date: Oct. 16, 2024

Related U.S. Application Data
(60) Provisional application No. 63/331,985, filed on Apr. 18, 2022.

(51) Int. Cl. A61B 1/07; A61B 1/04; A61B 1/05; A61B 10/04; G06N 20/00; G16H 30/40; G16H 50/20

(52) U.S. Cl. CPC A61B 1/07; A61B 1/041; A61B 1/043; A61B 1/05; A61B 10/04; G06N 20/00; G16H 30/40; G16H 50/20

(57) ABSTRACT

An imaging and biopsy device, including: a tethered capsule that is configured to be swallowed; a first optical fiber transmitting an electromagnetic radiation that at least partially impacts an anatomical structure; and a biopsy apparatus configured to collect tissue from the anatomical structure, the electromagnetic radiation at least partially or temporarily impacting the biopsy apparatus, and at least a portion of the first optical fiber and the biopsy apparatus being associated with the tethered capsule.

### CROSS-REFERENCE TO RELATED APPLICATIONS

The present application is based on and claims priority from U.S. Patent Application Ser. No. 63/331,985, filed on Apr. 18, 2022, the entire disclosure of which is incorporated herein by reference.

### BACKGROUND

Esophageal adenocarcinoma (EAC) is a deadly cancer that is preceded by a metaplastic change called Barrett's esophagus (BE). It has long been thought that endoscopic screening for BE followed by endoscopic surveillance can significantly decrease the mortality of EAC. This unfortunately has not been borne out, as the cost and inconvenience of conscious sedation prohibits endoscopy from being used as a population-based screening tool. BE screening may become possible in the future, owing to innovative swallowable tethered capsule endomicroscopes or cell sampling devices that can detect BE without requiring sedation. Yet, even if these capsules were to identify the large number of people in the US who have BE (˜15M), ongoing endoscopic surveillance of this group would be prohibitively expensive. However, if tissue biomarkers could be used to identify the ˜5% of those with BE who will develop EAC in their lifetimes, then endoscopic intervention could be given to those who need it most and those with low-risk BE would not require further follow up.

### SUMMARY OF THE INVENTION

Accordingly, new systems, methods, and apparatus for conducting non-sedated screening and biopsy are desirable.

In one embodiment, an imaging and biopsy device, including: a tethered capsule that is configured to be swallowed; a first optical fiber transmitting an electromagnetic radiation (e.g. UV, visible, or infrared light) that at least partially impacts an anatomical structure; and a biopsy apparatus configured to collect tissue from the anatomical structure, the electromagnetic radiation at least partially or temporarily impacting the biopsy apparatus, and at least a portion of the first optical fiber and the biopsy apparatus being associated with the tethered capsule.

In some embodiments of the device, the first optical fiber may include at least one of a single mode fiber (SMF) or a double clad fiber (DCF). In certain embodiments of the device, the tether may be configured to be torqueable. In particular embodiments of the device, the biopsy apparatus may include a cryobiopsy apparatus. In some embodiments of the device, the electromagnetic radiation may be optically coupled to an optical coherence tomography (OCT) system. Various embodiments of the device may further include a detector system optically coupled to the first optical fiber that generates an OCT image, where the biopsy apparatus may be at least partially visible in the OCT image.

In another embodiment, an imaging and biopsy device, including: a tethered capsule that is configured to be swallowed; a first optical fiber transmitting a first electromagnetic radiation and a second electromagnetic radiation that at least partially impact an anatomical structure; a second optical fiber receiving a third electromagnetic radiation that is emitted from the anatomical structure, a wavelength of the third electromagnetic radiation being different from a wavelength of the first electromagnetic radiation and a wavelength of the second electromagnetic radiation; and a biopsy apparatus configured to collect tissue from the anatomical structure, at least one of the first electromagnetic radiation or the second electromagnetic radiation at least partially or temporarily impacting the biopsy apparatus, and at least a portion of the first optical fiber, the second optical fiber, and the biopsy apparatus being associated with the tethered capsule.

In some embodiments of the device, the first optical fiber may include at least one of a single mode fiber (SMF) or a double clad fiber (DCF), and the second optical fiber may include a multimode fiber (MMF). In certain embodiments of the device, at least one of the first electromagnetic radiation or the second electromagnetic radiation may excite fluorescence in the anatomical structure to generate the third electromagnetic radiation. In particular embodiments of the device, the tether may be configured to be torqueable. In various embodiments of the device, the biopsy apparatus may include a cryobiopsy apparatus. In some embodiments of the device, the first electromagnetic radiation is optically coupled to an optical coherence tomography (OCT) system. Various embodiments of the device may further include a detector system optically coupled to the first optical fiber that generates an OCT image, where the biopsy apparatus may be at least partially visible in the OCT image. Some embodiments of the device may further include a fourth electromagnetic radiation and a fifth electromagnetic radiation remitted from the anatomical structure, where the fourth electromagnetic radiation may be transmitted to a reflectance spectroscopy system, and where the fifth electromagnetic radiation may be transmitted to a fluorescence spectroscopy system.

In one embodiment, a multimodality tethered capsule endoscopy biopsy system, including: a lens including a double clad fiber (DCF) and a multimode fiber (MIF) coupled thereto; an optical coherence tomography (OCT) system including an OCT light source, the OCT light source configured to transmit OCT light through a core of the DCF into the lens such that the OCT light is emitted from the lens toward a sample; an autofluorescence and diffuse reflectance (AF/R) spectroscopy imaging system including an AF/R light source, the AF/R light source configured to transmit AF/R light through the MMF into the lens such that the AF/R light is emitted from the lens toward the sample, and the AF/R spectroscopy imaging system configured to collect the AF/R light remitted from the sample via an inner cladding of the DCF; and a cryobiopsy system including a cryobiopsy probe configured to be placed in a field of view of the OCT system to obtain tissue from the sample.

Some embodiments of the system further include a sleeve into which the DCF and the MMF may be disposed. In particular embodiments of the system, the sleeve may include a channel disposed therein, wherein the cryobiopsy probe may be disposed within the channel to be placed in the field of view of the OCT system. In various embodiments of the system, the sleeve may include a strain relief at a distal end thereof, where a distal end of the channel may be coupled to the strain relief, and where the strain relief may include an opening in a lateral portion thereof through which the cryobiopsy probe extends into the field of view of the OCT system.

In some embodiments of the system, the lens may include a ball lens. Certain embodiments of the system may further include an extended spacer having a long axis, where the ball lens may be coupled to a distal end of the spacer, and where the DCF and the MMF may be coupled to a proximal end of the spacer. In various embodiments of the system, the DCF may be coupled to the distal end of the spacer in an orientation parallel to the long axis of the spacer, and the MMF may be coupled to the distal end of the spacer at an angle relative to the long axis of the spacer.

Particular embodiments of the system may further include a reflector located distal to the ball lens and disposed at an angle relative to the long axis of the spacer to direct light from the ball lens toward the sample. Some embodiments of the system may further include a motor coupled to the reflector, where the motor may be configured to rotate the reflector about the long axis of the ball lens.

Various embodiments of the system may further include a capsule coupled to the distal end of the sleeve via the strain relief, where the reflector, the ball lens, the spacer, and the motor may be disposed within the capsule. In certain embodiments of the system, the sleeve may include at least one of a torque coil or a braided sheath.

In some embodiments of the system, the AF/R light source may include a broad spectrum light source configured to provide light for diffuse reflectance imaging and at least one narrow band light source configured to stimulate autofluorescence in the sample. In various embodiments of the system, the broad spectrum light source may include an electro-optic modulator (EOM) configured to intermittently block or allow transmission of output from the broad spectrum light source to the MN/IF. In particular embodiments of the system, the at least one narrow band light source may include an LED light source configured to be switched on or off. In various embodiments of the system, the LED light source may include a plurality of LED light sources configured to emit light at 375 nm and 450 nm and configured to be switched at a rate of 100 kHz.

In some embodiments of the system, the AF/R light collected by the AF/R spectroscopy imaging system may include AF/R spectra, and clinical standard color autofluorescence imaging (AFI) images may be generated based on the AF/R spectra.

In particular embodiments of the system, the lens may include a GRIN lens.

In various embodiments of the system, the cryobiopsy system may further include a coolant and the cryobiopsy system may be configured to inject the coolant into the cryobiopsy probe.

In some embodiments, a method for multimodality tethered capsule endoscopy biopsy, including: providing a multimodality tethered capsule endoscopy biopsy system including a lens, an optical coherence tomography (OCT) system, an autofluorescence and diffuse reflectance (AF/R) spectroscopy imaging system, and a cryobiopsy system, the lens including a double clad fiber (DCF) and a multimode fiber (MMF) coupled thereto, the optical coherence tomography (OCT) system including an OCT light source, the AF/R spectroscopy imaging system including an AF/R light source, and the cryobiopsy system including a cryobiopsy probe configured to be placed in a field of view of the OCT system to obtain a biopsy tissue from a sample; obtaining, using the OCT system, OCT structural information from the sample by transmitting OCT light from the OCT light source through a core of the DCF into the lens such that the OCT light is emitted from the lens toward the sample; obtaining, using the AF/R spectroscopy system, AF/R information from the sample, including: transmitting AF/R light from the AF/R light source through the MMF into the lens such that the AF/R light is emitted from the lens toward the sample, and collecting the AF/R information remitted from the sample via an inner cladding of the DCF; analyzing at least one of the OCT structural information or the AF/R information to identify an area of interest in the sample; and extracting, using the cryobiopsy probe, the biopsy tissue from the area of interest for analysis.

In certain embodiments of the method, obtaining OCT structural information from the sample may further include: identifying, based on the OCT structural information, the cryobiopsy probe within the field of view of the OCT system. In some embodiments of the method, extracting the biopsy tissue from the area of interest may further include: guiding the cryobiopsy probe to the area of interest based on identifying the cryobiopsy probe within the field of view of the OCT system. In various embodiments of the method, the cryobiopsy system may further include a coolant and extracting the biopsy tissue from the area of interest may further include: injecting the coolant into the cryobiopsy probe.

In some embodiments of the method, transmitting AF/R light may further include: emitting light from a broad spectrum light source to provide light for diffuse reflectance imaging, and emitting light from at least one narrow band light source to stimulate autofluorescence in the sample. In certain embodiments of the method, the broad spectrum light source may include an electro-optic modulator (EOM), and emitting light from the broad spectrum light source may further include: intermittently blocking or allowing transmission of output from the broad spectrum light source using the EOM.

In particular embodiments of the method, the at least one narrow band light source may include an LED light source, and emitting light from at least one narrow band light source may further include: switching the LED light source on or off. In various embodiments of the method, the LED light source may include a plurality of LED light sources including a 375 nm LED light source and a 450 nm LED light source, and switching the LED light source on or off may further include: switching the plurality of LED light sources at a rate of 100 kHz. In some embodiments of the method, transmitting AF/R light may further include: alternately transmitting light from the broad spectrum light source, the 375 nm LED light source, and the 450 nm LED light source through the MMF toward the sample.

In various embodiments of the method, the AF/R information may include AF/R spectra, and analyzing at least one of the OCT structural information or the AF/R information to identify an area of interest in the sample may further include: generating a clinical standard color autofluorescence imaging (AFI) image based on the AF/R spectra, and analyzing the AFI image to identify the area of interest. In some embodiments of the method, analyzing the AFI image to identify the area of interest may further include: analyzing the AFI image to identify a region of the sample with an increased likelihood of including at least one esophageal cancer progression biomarker. In certain embodiments of the method, extracting the biopsy tissue from the area of interest for analysis may further include: preparing a histological sample of the biopsy tissue, and analyzing the histological sample to identify the at least one esophageal cancer progression biomarker.

In some embodiments of the method, analyzing at least one of the OCT structural information or the AF/R information to identify an area of interest in the sample may further include: generating, using a deep learning model, at least one metric related to BE dysplasia grade, an esophageal cancer progression biomarker anomaly, or aneuploidy based on at least one of the OCT structural information or the AF/R information, and identifying the area of interest in the sample based on generating the at least one metric.

In various embodiments of the method, analyzing at least one of the OCT structural information or the AF/R information to identify an area of interest in the sample may further include: analyzing the OCT structural information to determine at least one of a correlation of a derivative bandwidth (COD BW) or a group velocity dispersion (GVD), and identifying the area of interest in the sample based on determining at least one of the COD BW or the GVD.

In particular embodiments of the method, analyzing at least one of the OCT structural information or the AF/R information to identify an area of interest in the sample may further include: generating an OCT dysplasia and AFI carpet map based on the OCT structural information and the AF/R information, and identifying the area of interest based on the OCT dysplasia and AFI carpet map. In some embodiments of the method, identifying the area of interest based on the OCT dysplasia and AFI carpet map may further include: flagging a location on the OCT dysplasia and AFI carpet map to identify the area of interest, and extracting the biopsy tissue from the area of interest may further include: guiding the cryobiopsy probe to the flagged location, and extracting the biopsy tissue from the flagged location.

In various embodiments of the method, the MMW may be coupled to the lens at an angle α relative to an optical axis of the lens, and obtaining AF/R information from the sample may further include: transmitting the AF/R light from the AF/R light source through the MMF into the lens at the angle α such that a focal location of the AF/R light overlaps with OCT light and autofluorescence light returned from the sample.

In certain embodiments of the method, the multimodality tethered capsule endoscopy biopsy system may further include a reflector located distal to the lens and a motor coupled to the reflector, where the reflector may be disposed at an angle relative to an optical axis of the lens to direct light from the lens toward the sample, and where obtaining OCT structural information from the sample may further include: obtaining the OCT structural information from the sample while rotating the reflector, and where obtaining AF/R information from the sample may further include: obtaining the AF/R information from the sample while rotating the reflector.

### BRIEF DESCRIPTION OF THE DRAWINGS

Various objects, features, and advantages of the disclosed subject matter can be more fully appreciated with reference to the following detailed description of the disclosed subject matter when considered in connection with the following drawings, in which like reference numerals identify like elements.

FIG. 1 shows a partial view of a construction of a two-fiber ball lens apparatus. DCF—dual clad fiber, MMF—multimode fiber.

FIG. 2, panel A shows a prototype of the optics of a two-fiber ball lens, while panel B shows a cross-sectional OCT-AF image of a swine esophagus obtained ex vivo with the optics in panel A. The colored outer ring around the OCT image in panel B represents tissue autofluorescence (ex: 415 nm, 10 mW).

FIG. 3, panel A shows an optical diagram showing light emanating from the LED illumination unit through a multimode fiber (MMF) to the lens and onto a sample, then returning light from the sample S being collected by a dual clad fiber (DCF), where the core of the DCF is also used for OCT delivery/collection. Two separate detectors are employed: (1) a reflectance spectrometer and a custom fluorescence spectrometer that includes an emission filter, grating, and (2) an APD array; panel B shows a detailed view of the LED illumination unit which includes a broadband source coupled via an EOM to the output fiber. LED—light emitting diode, LP—linear polarizer, MMF—multimode fiber, SMF—single mode fiber DAQ—data acquisition card, DCF—dual clad fiber, DCFC—dual clad fiber coupler, EOM—electrooptical modulator, APD—avalanche photodiode.

FIG. 4 shows a construction of a multimodality tethered capsule endoscopy biopsy system including a cryobiopsy capsule and an interface with the cryobiopsy system (top panel) and cross-sectional views of two configurations of a sleeve (bottom panel).

FIG. 5 shows a system for detecting endogenous/exogeneous fluorescence spectroscopy and reflectance spectroscopy using a low loss optical shutter (EOM). LP—Linear polarizer, EOM—electro-optical modulator, BS—beamsplitter, M—mirror, DCF—dual clad fiber, DCFC—dual clad fiber coupler, MMF—multimode fiber, SMF—single mode fiber, LED—light emitting diode, APD—avalanche photodiode, DAQ—data acquisition card, F—filter, λ/4-quarter wave plate.

FIG. 6, panel A shows photograph of a construction of an OCT-TCE tethered capsule. Panel B shows a photograph of an unsedated study subject after swallowing the OCT-TCE device. Panel C shows an OCT-TCE image of the full thickness esophageal wall (tick marks=1 mm), showing circumferential BE mucosa. Panel D shows a 3D rendering of an OCT-TCE dataset acquired from a BE patient in vivo, demonstrating a focus of high-grade dysplasia (HGD), where the inset shows a cross-sectional view through the HGD tissue. Panel E shows a 3D fly-through view of white light data obtained by pulling a multimodality, RGB OCT-TCE device through a swine esophagus in vivo. Panel F shows an OCT-fluorescence TCE 3D fly-through view of a swine esophagus in vivo (ex: 650 nm; em: 700-800 nm) following topical methylene blue staining that was retained at the edges of biopsy sites (red arrows). The insets in panels E and F show samples of raw OCT data corresponding to the respective fly-through views.

FIG. 7 shows a photograph of an endoscopic AFI of BE showing magenta areas (white arrows) confirmed to be HGD by pathology.

FIG. 8, panel A shows a photograph of the cryobiopsy system and a 1.2-mm-diameter cryoprobe. Panel B shows a close up photograph of a cryoprobe showing the metal tip and throttle. Panel C shows a histological preparation of the human duodenum acquired by the cryoprobe in vivo, showing that the device is able to obtain a large, well-oriented tissue sample.

FIG. 9 shows a schematic of a construction of the MM-TCEB technology, where a capsule at the distal end is swallowed by a subject. AFI—autofluorescence imaging; OCT—optical coherence tomography; AF/R—autofluorescence and diffuse reflectance spectroscopy; F—optical fibers; L—lens; R—reflector; M—micromotor; Tip—cryoprobe metal tip.

FIG. 10, panel A shows a photograph of a prototype OCT tethered capsule endomicroscope with biopsy device (OCT-TCEB) Panel B shows a close-up photograph of the 11-mm-diameter capsule showing the 1.2-mm-diameter cryoprobe's tip emanating from the strain relief Panel C shows a cross-sectional OCT image of the esophagus of a living swine acquired with device, showing the cryoprobe's tip (orange spot pointed to by arrow) and a previously placed cautery mark (cyan/dotted region). Panels D-G show cross-sectional OCT images of the cryoprobe's tip (orange) in relation to the cautery mark (cyan/shaded regions) as the tether was torqued in vivo, showing the capacity of the capsule to rotate a full 360° until it is in contact with the cautery target (panel G). Panel H shows a hematoxylin and eosin (H&E) histological preparation of an esophageal cryobiopsy acquired by the OCT-TCEB's cryoprobe in a living swine. These results show that the TCEB concept is feasible and capable of acquiring high quality image-guided biopsies in vivo. White asterisk—motor wires; black asterisk—capsule wall. Bars, 1 mm.

FIG. 11 shows schematics of a construction of the MM-TCEB device (panel A) and tether cross-section (panel B, corresponding to dashed line in panel A).

FIG. 12, panel A shows a schematic of the optics and the Monte Carlo simulation. Panel B shows simulated OCT and AF/R illumination/collection spots at the capsule's wall.

FIG. 13 provides a schematic showing optical paths of OCT, fluorescence excitation (375 nm, 450 nm LEDs), reflectance excitation (400-700 nm LED), their respective detection channels, and system components. R—reflectance, FL—fluorescence, LP—linear polarizer, EOM—electro optic modulator, ExF—excitation filter, MMF—multimode fiber, SMF—single mode fiber, DCF—dual clad fiber, DCFC—dual clad fiber coupler, F—filter, PBS—polarizing beam splitter, M—mirror, EmF—emission filter, APD—avalanche photodiode, DAQ data acquisition.

FIG. 14, panel A shows an image of OCT+Evan's/AFI carpet map and panel B shows a MM-TCEB real time image, which provide an example MM-TCEB biopsy targeting user interface at the time of targeted biopsy acquisition. BE and Evan's criteria (yellow and cyan) were created from an OCT-TCE dataset, obtained from a patient with HGD in vivo, after automatic classification using CNNs developed by our group. AFI (magenta) regions are simulated.

FIG. 15 shows simulations illustrating Correlation of the Derivative (COD) bandwidth (COD BW) scatterer size estimation. Panel A shows backscattering Mie spectra for a 6-μm-diameter sphere. Panel B shows a graph showing the COD. The red dot indicates the first minima, and the red arrow indicates the COD BW. Panel C shows COD BW plotted against scatterer size showing the theoretical curve (jagged blue curve) and the 4th order approximation fitted curve (smooth red curve).

FIG. 16 shows COD BW OCT images of a normal human colon sample (Panel A) and a human colon adenocarcinoma specimen (Panel B). Insets show photographs of the corresponding histology. The color images were formed by overlaying the COD BW-determined scatterer sizes on the OCT image using an HSV pseudo-color scale where hue represents sizes ranging from 2 to 20 μm. Panel C shows distributions of COD BW mean scattering diameters for normal colon and colon adenocarcinoma specimens. Panel D shows an example of COD BW-based classification (the unknown is indicated by the green circle with an adjacent asterisk, * while the remaining red circles indicate cancer).

FIG. 17 provides a summary of data types.

FIG. 18 shows a proposed complete deep learning architecture based on multimodal CNNs and autoencoders followed by data fusion and a classification layer.

FIG. 19 shows an example of a system for multimodality tethered capsule endoscopy biopsy in accordance with some embodiments of the disclosed subject matter.

FIG. 20 shows an example of hardware that can be used to implement computing device and server in accordance with some embodiments of the disclosed subject matter.

FIG. 21 shows an example of a process for multimodality tethered capsule endoscopy biopsy in accordance with some embodiments of the disclosed subject matter.

### DETAILED DESCRIPTION

In accordance with some embodiments of the disclosed subject matter, mechanisms (which can include systems, methods, and apparatus) for conducting non-sedated screening and biopsy are provided. Thus, the present disclosure provides various embodiments of swallowable tethered capsule screening tools as well as related systems and methods.

One of the biggest challenges in reducing the mortality of esophageal adenocarcinoma has been screening for people at high risk for developing this deadly cancer. Disclosed herein is a swallowable tethered capsule screening tool that obtains targeted biopsies of regions of the esophagus that harbor molecular abnormalities that confer risk for developing cancer. Analysis of these biopsies may determine who needs intervention, intercepting esophageal cancer before it has a chance to form.

Fewer than 5% of patients with gastroesophageal reflux disease (GERD) are screened for the presence of Barrett's Esophagus (BE) due to limitations of sedated endoscopy, the current standard of care. Consequently, many undetected BE patients eventually progress to esophageal cancer, and the incidence and mortality of esophageal cancer continue to rise.

An optical coherence tomography (OCT) tethered capsule endoscopy (TCE) platform has been previously demonstrated for unsedated optical biopsy of patients' upper GI tract in primary care and physician office settings. This platform enables clinicians to use subsurface features to identify suspected lesions. In this study, we demonstrate an improved OCT TCE platform capable of unsedated biopsy extraction from such suspected regions, allowing subsequent histopathology and immunohistochemistry (IHC) evaluation to confirm disease diagnosis and assess esophageal cancer progression biomarkers such as P53 mutations.

In certain embodiments, a swallowable capsule's tether and proximal cap disclosed herein have been modified to include a working channel. This channel allows a narrow cryobiopsy tool to be guided to the suspected lesion under OCT guidance. When a refrigerant is activated, tissue sticks to the probe in a “tongue on a flagpole” fashion; the tissue can then be extracted by retracting the cryobiopsy tool.

As discussed further below, initial swine studies (n=2) have been conducted where a torqueable tether rotated the cryobiopsy probe to a cautery mark; the tissue was then frozen to the cryoprobe and subsequently extracted. Extracted tissue was processed with standard histology methods, maintaining its architectural integrity for diagnostic and IHC assessment.

Embodiments of the disclosed procedures for biopsy extraction may lead to a new screening approach that would be appropriate for a broader, population-level screening. This would improve patient outcomes and decrease healthcare costs.

### Optical System Background

A typical multimodality optical system includes illumination and detection optics for each modality. This can require fast illumination/detection switching for accurate co-registration of different data streams. Sources can include lasers, lamps, LEDs, or other components having sufficient power based on the SNRs required. One useful modality is broadband reflectance. Advances in incoherent LED technology (e.g., automotive headlights motivating higher power broadband sources) and their low cost mean they increasingly present an attractive option for broadband light delivery. Problematically, the way broadband light is generated limits how quickly these LEDs can be switched on and off. Typical LEDs have a narrow bandwidth (typically <50 nm) and can be modulated to speeds of around 100 kHz. It is possible to convert a typical 450 nm LED into broadband by overlaying a phosphor on the array. While these phosphors help to convert a narrowband LED (e.g. at 450 nm) into a broadband source, the slow response time of the phosphors makes them less suitable for use as high-speed switched sources.

Dopants can further increase the spectral uniformity of the light distribution. The phosphor has a fluorescence lifetime that makes modulation impractical >5 kHz. Consequently, new illumination modulation techniques are needed to use broadband LEDs with high switching speeds.

High-speed illumination switching can make it necessary to modify downstream detection optics. In some cases, sources for one modality (e.g., reflectance) have a bright signal that can saturate and potentially damage the detectors from another modality (e.g., fluorescence). The speeds required may be on the order of 100 kHz (and may be within a range of 50 kHz to 200 kHz), making mechanical switches unsuitable. It is desirable that high-speed optical shutter/switching techniques be developed which isolate these detectors.

Accordingly, optical systems for next-generation imaging combining multi-modalities should be capable of high-speed broadband switching on both illumination and detection arms for techniques that cannot be used in parallel.

### Two-Fiber Ball Lens

Disclosed herein are embodiments of a two-fiber ball lens that can be used to deliver/collect light from tissue in a capsule/catheter-based device. The decoupling of the illumination and detection light paths vastly reduces the autofluorescence generated in the fiber.

In particular, dual clad fiber (DCF) fluorescence systems use either the inner core or inner cladding of the DCF to deliver fluorescence excitation and almost all systems collect fluorescence through the inner cladding. Autofluorescence can be generated when the silica, impurities in the silica, or other fiber materials such as certain plastics are excited. The autofluorescence is typically generated by excitation light and not emission light, given that excitation light intensity is typically much greater than that of emission light (>1000×). When excitation light passes through the silica-based fiber, the silica autofluorescence, which often overlaps with the emission pass-band, leads to both extra signal shot noise and a reduction in usable detector range. This can occur when light returns through the same channel it was emitted from, and this is often caused by excitation light being transmitted through the cladding followed by collection light also being transmitted through the cladding. Optical fiber autofluorescence can also be detected when the light field excites fluorescence in the cladding or when fluorescence generated in one channel crosses between channels, typically when excitation is through the core followed by collection through the cladding. However, by separating the two channels (excitation and emission) into two separate fibers, autofluorescence light does not return to the detector.

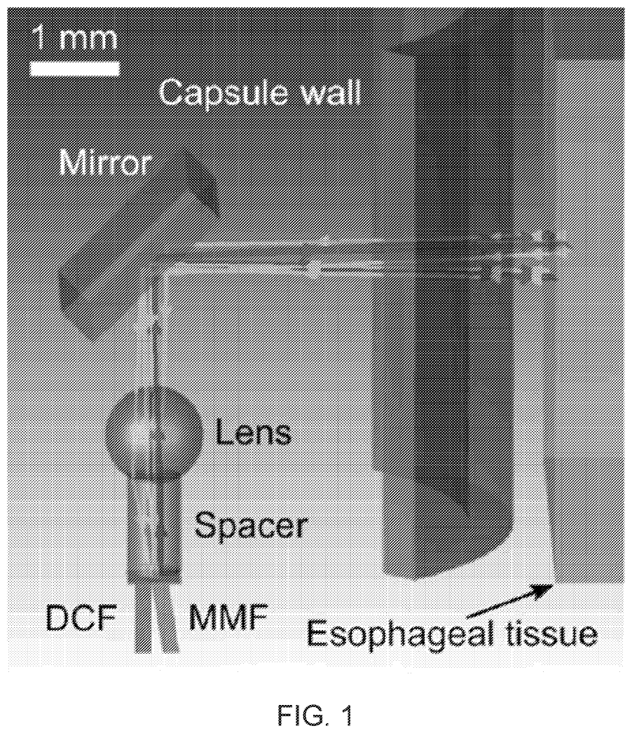

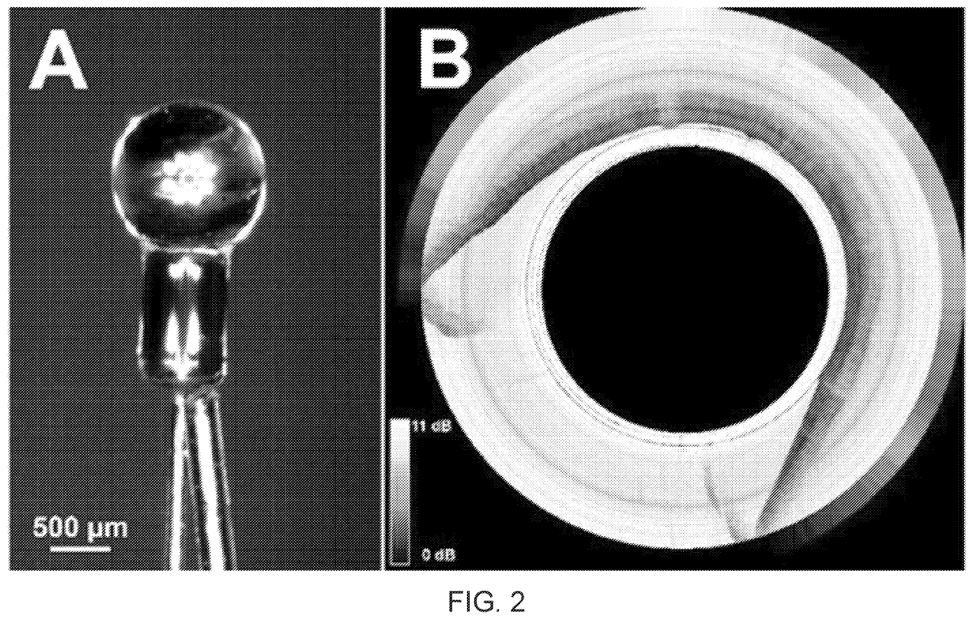

A two-fiber ball lens can be made by affixing two fibers (e.g. dual clad, multimode) to a lens such as a ball lens or a grin lens, or to another optical element. The fibers can be attached using epoxy, heat fusing, or other methods. In the case of the constructed prototype, heat fusing was used to fuse a glass rod (although plastic may also be suitable) to one of the fibers. The second fiber was then epoxied next to the first fiber. Light can be sent via the multimode fiber and focused onto tissue via the ball lens; reflectance and fluorescence emission can be collected via the inner cladding of the dual-clad fiber. It is also possible to deliver/collect with the multimode fiber and replace the dual clad fiber with single-mode fiber. In one embodiment, the capsule optics of an 8 mm capsule were used to create a testing setup (FIG. 1). The setup (FIG. 2A) was used to acquire an optical coherence tomography slice co-registered with a fluorescence image (FIG. 2B).

### Optical System

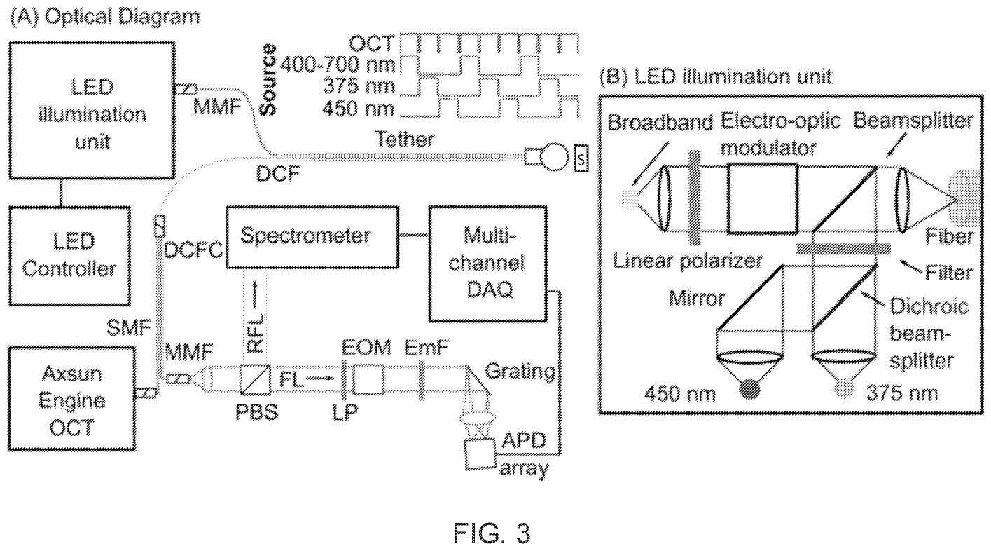

Disclosed herein are embodiments of an optical system configured to enable >100 kHz illumination pulsing of both narrow and broadband LEDs. On the detection side, to electronically rapidly switch detection arm light paths protecting detectors a voltage variable waveplate (electro-optic modulator, EOM) may be used. In the optical system shown in FIG. 3A, light is guided from the illumination arm into a multimode fiber and then collected by the inner cladding of a dual clad fiber and detected as described below.

### Optical System—Illumination

Various embodiments of the disclosed system may employ a free space LED configuration to multiplex different optical channels into a single channel. Light from a broadband LED can be combined with narrow band (non-phosphor based) LEDs via a series of mirrors and beam splitters (e.g. see FIG. 3B). Prior to being combined, an electro-optic modulator (voltage variable wave plate, Inrad Optics, PKC21-SG25) acts as a switch for the broadband (reflectance detection) path. In the fluorescence light path, filtering is used to clean up excitation light. The narrow band LEDs (375 nm, 450 nm shown) are pulsed at 100 kHz speeds electronically. The broadband light is pulsed using a linear polarizer and an electro-optic modulator at speeds up to 250 MHz (note that the benefits that are realized using a phosphor-based LED may be generalized to the use of any arbitrary light source including LEDs needing >100 kHz modulation, lamps, lasers, etc.). The extinction ratio (ratio of input light to output) is limited by that of the electro-optic modulator EOM (>1000:1 is typical). The EOM can have an aperture of varying sizes, limited by the optical properties of the crystal (note that Kerr gates, which have previously been reported for use in high-speed fluorescence detection, can also be used for this purpose).

More broadly, the issues of high-speed light switching have been recognized in the growing field of so-called “LiFi” communications (using modulated LED light for communication with electronics), where one solution has been to use bandpass filtering. A phosphor-based LED uses a ˜450 nm driving LED to excite lower wavelength phosphorescence. This means a narrow band around 450 nm can be used to reliably obtain a well-modulated signal. While this has its advantages, the amount of light that can be collected is limited. It is not unreasonable to envisage a miniaturized EOM on top of an LED array capable of high speed full-visible spectrum modulation.

### Optical System—Detection

Light entering the detection system from a dual clad fiber may be separated into single mode (inner core) and multimode (inner cladding) channels. The single mode path may be used for both OCT incident light and OCT reflected light, while the multimode path is used for the collection of fluorescence and reflectance light. Light returning from the multimode port is then separated by a polarizing beam splitter into two channels, reflectance (which can be sent to a spectrometer or other grating-based unit) and fluorescence.

Prior to light entering the fluorescence channel there is an additional EOM and linear polarizer. The reason for this is that the channels are pulsed, and as reflectance light is much higher intensity than fluorescence illumination it is necessary to protect the fluorescence detector during reflectance imaging (however, it is not a requirement that the detectors be reflectance and fluorescence, but it is preferable that one detector is not receiving light while another is). The fluorescence detector is a grating and 16 element APD array. A multichannel data acquisition card digitizes light from the fluorescence and reflectance detectors.

Accordingly, both the two-fiber ball lens configuration and the procedures for coupling a white light LED with an electro-optic modulator (EOM) for switching, as well as the EOM used on the detection arm to protect more sensitive fluorescence detectors, provide novel approaches to solve one or more problems disclosed herein.

### Capsule Systems

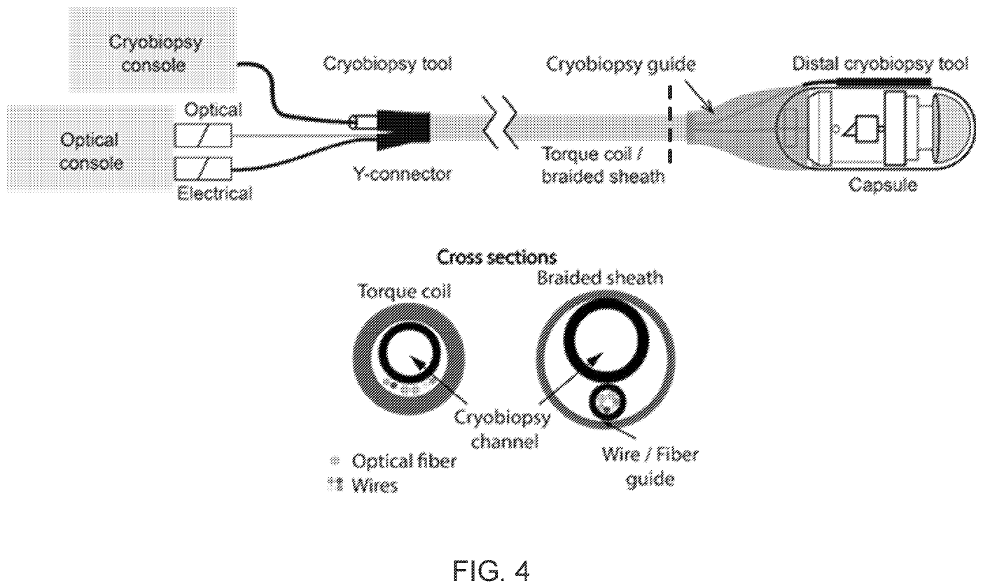

Various embodiments of a capsule may include a tether, wires, fiber, proximal connectors, and/or a distal capsule-like body. FIG. 4 shows an embodiment of a multimodality tethered capsule endoscopy biopsy system including a cryobiopsy capsule and an interface with the cryobiopsy system (top panel) and cross-sectional views of two configurations of a sleeve (bottom panel). A Y-connector couples optical and electrical connections as well as any appropriately sized tool such as a cryobiopsy tool or any other appropriately sized tool to be guided by the working channel to the distal capsule. The working channel and wires and fibers may be enclosed by a sleeve which can include a torque coil and/or a braided sheath (see cross sections of each possible enclosure in bottom panel, the location of the cross-sectional views corresponding to the dashed line in the top panel). Distally, the probe may be guided to the tissue via a rigid/flexible guide or strain relief. The cryobiopsy tool may include a commercially available refrigerant along with guides for the refrigerant and suitable electronics.

The braided sheath or torque coil may be used to allow torquability of the capsule. The distal Cryobiopsy guide can either be a rigid structure or flexible strain relief. The capsule contains one/two fibers terminated with a ball lens or grin prism. The tether may include either a working channel and fibers/wires out of a channel, or two channels one for the Cryobiopsy tool and one to guide the wires/fiber.

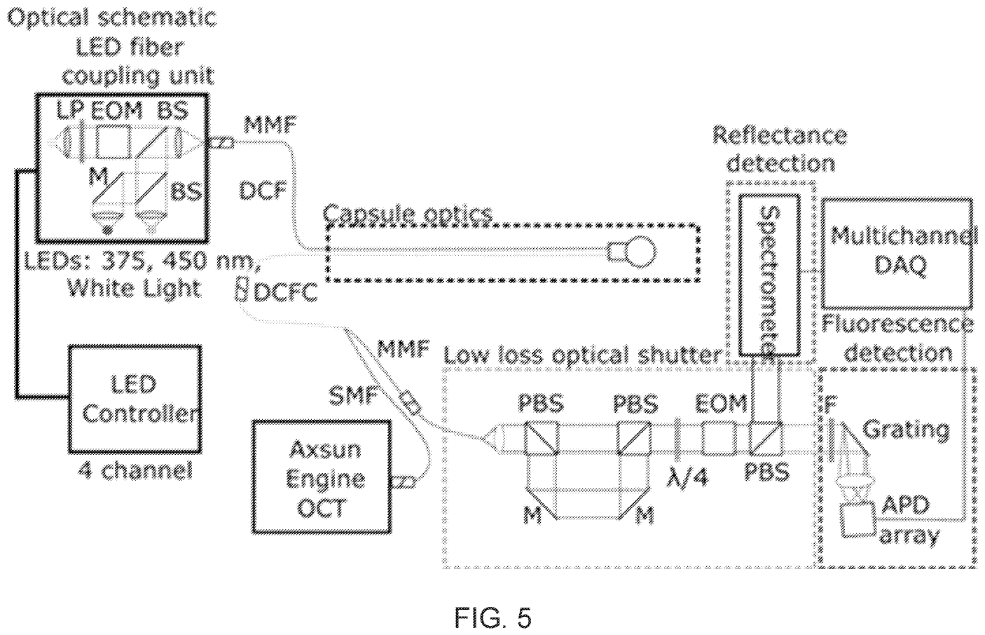

An embodiment of an optical system for detecting endogenous/exogeneous fluorescence spectroscopy, and/or reflectance spectroscopy using a low loss optical switch is shown in FIG. 5. Light is guided from a source unit comprising 3 light-emitting diodes (LEDs) and other components which control switching (including electro-optical modulators, beam splitters) and relaying light (mirrors/lenses). The LEDs are controlled by an LED controller. Any sources can be used (e.g. centered at 300-400 nm (bandwidth (BW): <˜20 nm), 400-450 nm (BW: <˜20 nm), and/or 350-750 nm (BW: 300-400 nm)) and, in addition to LEDs, can include xenon lamps and/or lasers. The light is then relayed to tissue via a multimode fiber (MMF), although a dual clad fiber (DCF)/W-type fiber can also be used. Light which returns from the tissue via reflection or fluorescence is then captured in the DCF which contains an inter channel to guide OCT light (discussed later), and an outer channel collects the returning fluorescence. Light is then separated out of the DCF using a DCF coupler (DCFC) and guided via a multimode fiber to a free space, low loss collection unit. Polarization optics are used to efficiently convert unpolarized light to polarized, and switch between reflectance/fluorescence detection.

There have been reports of methods of efficiently converting unpolarized light to single axis polarized using polarizing beam splitters (PBS) to split the light into horizontal linearly polarized (HLP) and vertically linearly polarized/VLP, then recombining the light and passing through a λ/4 waveplate to convert the light to left and right circularly polarized light. When these waves add, the result is horizontally linear polarized light. This polarized light then goes into an electro-optical modulator (EOM, voltage-variable waveplate) which can be controlled in high speed to change the polarization states. Depending on the state which exits the EOM a polarizing beam splitter either relays the light to a reflectance spectrometer or a fluorescence spectrometer. Both spectrometers use lenses to collimate/partially collimate the light, gratings and either cameras, photo multiplier tubes (PMT) or PMT arrays or avalanche photo diodes (APD) or APD arrays. Optical coherence tomography light from a system which controls light generation and detection is coupled into the inner core via the DCFC and guided through the inner channel of one of the two-fibers to the catheter's distal tip where reflected light is collected in the same channel and relayed back the OCT system. Data is captured by a multichannel data acquisition card and relayed to a personal computer (not shown).

In certain multi-modality capsule systems, the light has been delivered to the tissue via the inner core of a dual clad fiber, which is pigtailed to a ball lens, and returning light guided by the ball lens is collected via the cladding. However, autofluorescence generated by the fiber itself can lead to a background signal that contributes additional noise. This is further compounded using 375 nm-450 nm excitation where the fiber's optical absorption and fluorescence background are higher. Thus, designs are needed which can transmit fluorescence excitation light and collect emission with a low fiber background increasing the SNR/dynamic range. As a result, a new type of ball lens has been desired which can reduce or eliminate the high fluorescence background to recover SNR, which would enable improved low-light imaging at all wavelengths and enable UV wavelengths to be used for fiber-based excitation/collection.

Esophageal adenocarcinoma (EAC) is a deadly cancer that is preceded by a metaplastic change called Barrett's esophagus (BE). It has long been thought that endoscopic screening for BE followed by endoscopic surveillance can significantly decrease the mortality of EAC This unfortunately has not been borne out as the cost and inconvenience of conscious sedation prohibits endoscopy from being used as a population-based screening tool. BE screening may become possible in the future, owing to innovative swallowable tethered capsule endomicroscopes or cell sampling devices that can detect BE without requiring sedation. Yet, even if these capsules were to identify the large number of people in the US who have BE (˜15M), endoscopic surveillance of this group would be prohibitively expensive. If we could use tissue biomarkers to identify the ˜5% of those with BE who will develop EAC in their lifetimes, then endoscopic intervention could be given to those who really need it, and those with low-risk BE would not require further follow up.

Recognition of this need has motivated the field to identify BE progression biomarkers derived from esophageal tissue samples obtained by autofluorescence/reflectance-targeted endoscopy. This research has identified biomarkers such as aneuploidy and aberrant p53/cyclin A expression as strong predictors of BE progression. Unfortunately, the only way to target and obtain these tissues today is through sedated endoscopy With the modifications proposed here, a new BE screening technology that we have developed called optical coherence tomography (OCT) tethered capsule endomicroscopy (TCE), could enable targeted biopsy without sedation. OCT-TCE obtains 3D microscopic images of the entire esophagus in unsedated subjects, accurately identifies BE, and has been successfully used by nurses and technicians in primary care settings Here, we disclose advances in OCT-TCE for targeted biopsy by adding autofluorescence and reflectance spectral imaging technology which can help identify tissues which may be enriched in molecular alterations associated with BE progression risk. The new capsule may also have a cryobiopsy mechanism for acquiring targeted tissue samples under real time image guidance. In various embodiments, this multimodality TCE with biopsy (MM-TCEB) device will be developed and it will be demonstrated that the device works as intended in a preliminary study of 20 BE patients. A further clinical study will be conducted in 100 unsedated BE patients to demonstrate that MM-TCEB can be used to collect tissue that can be used to identify BE progression biomarkers as effectively as sedated endoscopy. Subsequent embodiments will entail development of image analysis and deep learning algorithms to mine this data, uncovering new relationships between OCT, autofluorescence, and reflectance spectroscopy and tissue-derived BE progression biomarkers. By acquiring targeted biopsies using a swallowable tethered capsule in unsedated subjects, MM-TCEB can become a powerful technique for obtaining esophageal tissue samples for BE progression biomarker discovery, validation, and ultimately population-based screening.

Esophageal columnar cell metaplasia or Barrett's Esophagus (BE), which is found more often in people with gastroesophageal reflux disease (GERD), is the precursor of esophageal adenocarcinoma (EAC), a cancer with a national incidence of 10,000/yr and a 5-year mortality rate of ˜20%. The current standard of care entails endoscopic screening for BE in subjects with symptomatic GERD, continued surveillance, and treatment of dysplastic BE. Unfortunately, this strategy has failed to significantly reduce EAC incidence and mortality. One major barrier has been the lack of an inexpensive, well-tolerated, and accurate BE screening method. The outlook has improved somewhat due to newer swallowable tethered capsule devices that either sample superficial cells or capture microscopic images of the entire esophagus without requiring sedation. Nevertheless, identifying everyone with BE is not tenable: because of the high prevalence of BE in the general population, the low likelihood of BE progressing to EAC, and the high cost and inefficiencies of endoscopy, surveillance of this large cohort would overwhelm the healthcare system. Thus, work has been directed at identifying biomarkers that help predict who with BE will progress to EAC in the future. This “holy grail” of biomarkers will enable actionable capsule-based BE screening while improving the cost/efficacy of follow up through precise diagnosis and early intervention only for those who are at risk of developing EAC.

An area of active research, tissue-derived biomarkers such as mutations, aberrant p53 expression, cyclin A overexpression, aneuploidy, gene methylation, and clonal diversity, among others, have been identified as promising predictors of BE progression. Tissue for detecting the presence of these biomarkers has primarily been obtained by sedated endoscopic biopsy, with recent studies employing endoscopic autofluorescence imaging (AFI) to target areas likely to be enriched with molecular/genetic anomalies. However, endoscopy is undesirable for BE screening, because it is too expensive and is inconvenient for use in large populations.

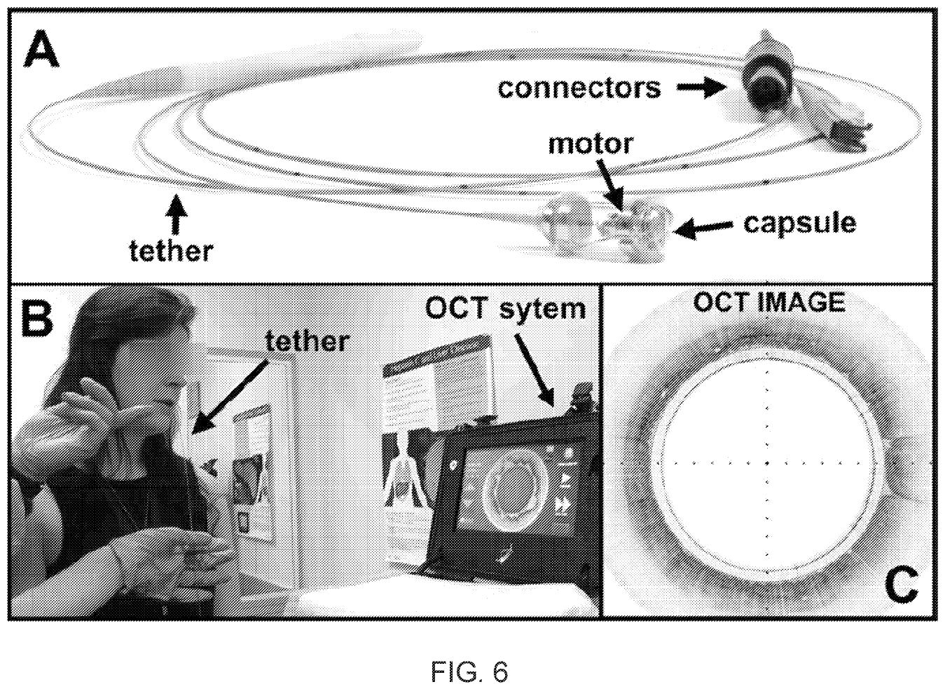

Our group has developed an optical coherence tomography (OCT) tethered capsule endomicroscopy (TCE) device (FIG. 6A) which conducts three-dimensional esophageal microscopy in unsedated patients, has a high accuracy for detecting BE, is well tolerated, and can be administered by nurses/technicians in a primary care setting (FIGS. 6B-6F). However, a further improvement that would enhance this device would be the ability to identify the most high-risk tissues in real time and biopsy them for assessment of BE progression biomarkers. This gap may be bridged by advancing OCT-TCE technology through the addition of autofluorescence/reflectance (AF/R) spectral imaging, from which AFI can be derived, to highlight regions which may have elevated molecular/genetic aberrations, and a tiny cryobiopsy probe (cryoprobe) that obtains high quality tissue samples from these locations. This multimodal TCE biopsy (MM-TCEB) device will be used in clinical studies to obtain OCT/AFI-targeted BE tissues from which progression-associated biomarkers will be derived. Data from these studies will be mined to extract additional OCT and AF/R metrics that are correlated to these biomarkers, improving the precision of image-based biopsy targeting and opening the possibility of tethered capsule BE progression screening without requiring tissue sampling.

### Multimodality TCE Biopsy (MM-TCEB) Technology for Targeted Sampling of BE Tissue.

To enrich biomarker yield, our OCT-TCE capsule/system will be augmented to collect both OCT and AF/R spectral data. To avoid background fiber fluorescence, the tethered capsule will utilize a two-fiber design where the core of a double clad fiber (DCF) will carry OCT (e.g. at 1310±75 nm) light and its inner cladding will collect remitted AF/R light (400-700 nm). A separate multi-mode fiber (MMF) will excite AF at two narrow band wavelengths (ex: 375 and 450 nm) to capture metabolic (optical redox) and other intrinsic molecular information. Clinical standard color AFI images will be computed from AF/R spectra. This will be done using standard methods of either spectral binning using bins 400-500, 500-600, and 600-700 nm for blue, green, and red, respectively, or by applying standard RGB filter transmission profiles to raw spectra. The tether/capsule will contain a channel through which a small cryoprobe can be inserted so that it is visible by OCT when it exits at the capsule. For abnormal regions seen by OCT/AFI, the tip of the cryoprobe will be rotated to the target site and frozen; the adhered tissue will be pulled back through the capsule/tether. MM-TCEB will be tested in a clinical study (n=20) to show that it can reproduce clinical standard AFI and acquire tissue samples that are comparable to endoscopic forceps biopsies.

### Determination of the Capacity of MM-TCEB to Acquire Targeted Tissue Samples Containing BE Progression Biomarkers In Vivo

Clinical studies will be conducted that use the MM-TCEB device to acquire co-registered OCT and AF/R spectral images of the entire esophagus in 100 unsedated patients with BE. Biopsies will be taken from BE locations identified as potential regions of concern by real-time OCT/AFI TCE. Established BE progression biomarkers (p53 and cyclin A anomalies and aneuploidy) will be extracted from image-guided cryobiopsy tissue samples and compared to histologic dysplasia grade, a proxy for BE progression.

### Development and Validation of MM-TCE Imaging Biomarkers for BE Progression.

The predictive capacity of MM-TCEB depends on the molecular aberration yield of tissues sampled from the esophagus. To improve biopsy targeting enrichment, we will develop/validate novel image analysis and deep learning algorithms using the acquired OCT and AF/R data; histology/biomarkers from corresponding cryobiopsies will be utilized as standards for comparison. Light scattering spectroscopy (LSS) and OCT correlation of the derivative (COD) will be developed/validated to quantify epithelial aneuploidy and an innovative deep learning pipeline will be created to generate metrics related to BE dysplasia grade, p53/cyclin A anomalies, and aneuploidy.

Even though the current endoscopic standard of care for BE has been practiced for 40+ years, EAC incidence and incidence-based mortality continue to rise. One critical shortcoming of today's BE management paradigm is upper endoscopy itself, which is too invasive and expensive for population-based BE screening. Indeed, the majority (90%) of those with GERD and nearly everyone else are not screened for BE, largely due to the cost and inconvenience of conscious sedation used in most endoscopic procedures. Resultantly, endoscopy referral rates in general/primary care practices are low and most people with BE go undiagnosed. This underutilization of BE screening is likely the main reason why ˜90% of EACs arise in patients without a prior diagnosis of BE. Yet, even if this failing were to be overcome with a less invasive screening test, identification of all BE patients in the population would create a new problem of scale. EAC incidence rates in BE patients are about 0.33% per year; thus, only ˜5% of those diagnosed with BE will ever develop esophageal cancer in their lifetimes. Given that ˜5% (˜15M) of the US population has BE, endoscopic surveillance of this large cohort would overwhelm our endoscopy capacity and be financially unsustainable.

This problem is exacerbated by the limitations of endoscopic surveillance for monitoring the neoplastic status of BE patients. The current dogma is that BE progresses through a series of genetic/epigenetic alterations that curb normal epithelial cell maturation, reflected in histomorphologic entities that evolve from non-dysplastic BE (NDBE) to low grade dysplasia (LGD), high grade dysplasia (HGD), intramucosal carcinoma (IMC) and, eventually, invasive EAC. Dysplasia is a powerful predictor of BE progression to EAC, motivating endoscopic surveillance to uncover its presence and eradicate it by ablative therapy. Unfortunately, dysplasia is generally not readily visible by white light video endoscopy, which can lead to sampling error, erroneous diagnoses, and even missed cancer diagnoses in some cases. The recommended Seattle Protocol (biopsy of visible lesions and 4-quadrant random biopsy sampling every 2 cm along the BE segment), which reduces sampling error but still omits 95% of the BE segment, is time-consuming and costly and not robustly performed in most endoscopy clinics. Further compounding the problem is low pathologist interobserver agreement for dysplasia diagnosis, especially for LGD. The subjectivity and uncertainty regarding BE dysplasia diagnosis dictates surveillance at intervals that are overkill for most, as only a small percentage of BE patients will ever develop EAC. There are no randomized controlled trials that show improved outcomes with endoscopic surveillance and most analyses indicate that the cost-effectiveness of surveillance is questionable.

Nevertheless, this situation could be remedied at least in part through the use of biomarkers that predict who with BE is likely to progress to EAC and a non-endoscopic device for obtaining tissue for identifying the presence of these biomarkers in unsedated subjects that is sufficiently inexpensive and convenient to use to enable screening of large populations. These advances would allow us to capture a much larger percentage of people who are at risk of developing EAC, directing them to undergo endoscopic intervention when needed. Conversely, BE patients who are at low risk for progression would not need to undergo surveillance or intervention, eliminating the high cost of millions of unnecessary endoscopies.

### BE Progression Biomarkers

For these reasons, the identification of biomarkers for predicting BE progression has emerged as an active field of current research. Studies have shown that many molecular/genetic alterations in BE, including aberrant p53 expression, cyclin A overexpression, Ki-67 overexpression, epithelial lectin adhesion, miRNAs, DNA methylation, aneuploidy, mutational load, and/or clonal diversity, may improve the diagnosis of dysplasia and/or predict the likelihood of NDBE progression to high-grade dysplasia (HGD) or EAC. Perhaps the most well studied BE progression biomarker is p53 over/under expression. Aberrant p53 expression can be evaluated by immunohistochemistry (IHC), making it a relatively straightforward assay to conduct. A new meta-analysis examining over forty p53 IHC BE studies has found that abnormal p53 expression had a cohort study odds ratio (OR) of 4-6 and a relative risk (RR) of 14-17 in case control studies for progression. Surface epithelial expression of cyclin A can also be assessed by IHC, which is elevated in increasing grades of dysplasia, and has been shown to be associated with BE progression. Aneuploidy, measured by flow/image cytometry, indicative of extensive DNA changes due to genomic instability, is another key tissue biomarker that was recently found to be superior to p53 and cyclin A for predicting BE progression beyond 4 years. The use of biomarker panels is a growing trend; recent research showed that a combination of aneuploidy, cyclin A, and p53 expression had 95% accuracy for diagnosing HGD/EAC and aneuploidy+p53 had an AUC of 0.68 for BE progression to HGD/EAC.

While results from research done to date to identify BE progression biomarkers have been promising, this work is still at an early stage, as most studies have been small with modest positive predictive values (˜20-60%). Additional research is needed to conduct larger studies to validate these biomarkers and identify additional biomarkers/panels/algorithms that more precisely predict progression. Undoubtedly, new tools that increase the pace of BE progression biomarker discovery and clinical validation would be welcome additions to this field.

### Comparison to Current Tissue Biomarker Collection Approaches

Though liquid biopsy is noninvasive and relatively straightforward, it is not likely to yield high levels of BE progression biomarkers since the metaplastic epithelium is confined within an uncompromised basement membrane. Measuring volatile organic compounds (VOC) in exhaled breath is another noninvasive technique that has been investigated for the detection of BE with promising preliminary results that merit further validation, although there is no evidence yet that VOCs are capable of informing on BE progression. Most BE progression biomarkers studied thus far have been derived from BE tissue. The current methods for collecting BE tissue biomarkers include endoscopic biopsy, with or without AFI targeting, and tethered, encapsulated cell sampling devices. While the former provides excellent tissue samples for biomarker discovery, as detailed above endoscopy is impractical for population-based BE progression screening.

Cell sampling via swallowable, tethered capsules that turn into sponges in the stomach and are pulled back through the esophagus (e.g. Cytosponge, EsophaCap) and encapsulated balloon devices (e.g. EsoCheck) are well positioned for high risk BE screening because they are relatively simple and do not require sedation. In one recent prospective study, multivariate regression was performed based on patient characteristics (e.g. age, waist-to-hip ratio, and endoscopic BE segment length) and glandular atypia, p53 abnormality, and Aurora kinase A positivity, derived from Cytosponge samples. Regression results were used to segment patients into low, moderate, and high risk categories for harboring HGD/IMC, as determined by subsequent endoscopy. Most validation patients (>50%) ended up in the moderate risk group, but for the low-risk group, the probability that HGD/IMC was not found was high (96%), suggesting that these patients could potentially forego endoscopic surveillance. Although a promising start, the algorithm did not directly evaluate BE progression, produced many indeterminant results, and used data from follow up endoscopy that was not measured by the device.

Encapsulated cell sampling tests such as these have significant upsides, namely they use simple and well-tolerated devices that do not require sedation and they safely harvest pan-esophageal superficial tissue samples from which many potential BE progression biomarkers can be derived. Nevertheless, they also have limitations that will likely limit their accuracy for predicting BE progression. Since these devices do not use imaging, they often sample the gastric cardia, which can also be metaplastic, resulting in false positives. They indiscriminately acquire cells from large portions of the esophagus and thus are not targeted to enrich biomarker collection, potentially causing small tissue foci containing molecular/genetic aberrations to be missed or overwhelmed by a much larger background signal. This latter point is particularly relevant as recent research has revealed that small BE regions at the gastroesophageal junction (GEJ) are common and have much higher malignant potential than previously thought. Encapsulated cell sampling devices also cannot directly quantify relevant parameters such as BE length, spatial heterogeneity, and the location of esophageal abnormalities for future intervention. Because they only scrape off superficial epithelium, the sampled tissue obtained with such devices may not be ideal for evaluating architectural maturation, which is a critical feature used by pathologists to diagnose dysplasia. Likewise, they may undersample molecular/genetic abnormalities in metaplastic crypts where stem cells that give rise to clonal expansions reside and are unable to acquire metaplastic cells under squamous epithelium, which is a common finding in BE.

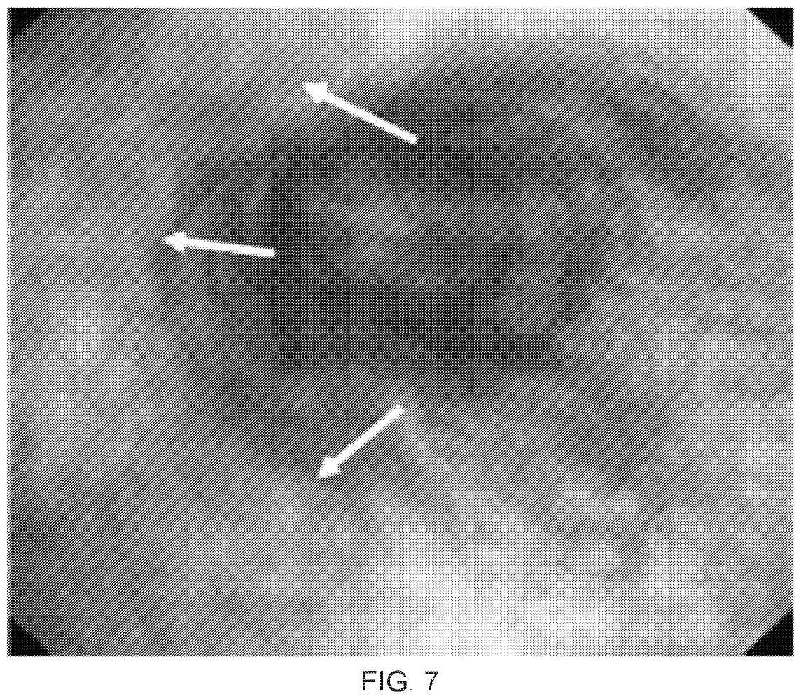

Accordingly, the present disclosure presents the development and validation of alternative minimally invasive tethered capsule approaches that use image-guided biopsy to overcome the limitations of endoscopy and encapsulated cell sampling devices for biomarker tissue sampling. The multimodality TCE with biopsy (MM-TCEB) device will employ label-free, three-dimensional (3D) microscopic morphologic (OCT) and molecular/chemical imaging (AF/R spectroscopy), from which clinically established AFI will be derived, to target cryobiopsy tissue sampling. Once a region within the esophagus that has been identified as being enriched has been identified by OCT/AFI TCE, a small, flexible cryoprobe (FIG. 8B) will be inserted through the tether and guided around the capsule under OCT image guidance so that its active metal tip is located over the targeted region. The tip will be frozen, adhering a tissue sample to the cryoprobe. The cryobiopsy will then be retrieved by withdrawing the cryoprobe through the tether and the tissue sent for molecular analysis to evaluate BE progression risk biomarkers. By enabling targeted biopsies to be acquired using a tethered capsule that is swallowed by unsedated subjects, MM-TCEB will become a powerful technique for obtaining esophageal tissue samples for BE progression biomarker discovery, validation, and ultimately population-based screening.

A swallowable tethered capsule for image-targeted biopsy of BE progression biomarkers is new and will be transformative for the field. MM-TCEB's image-targeted biopsy enrichment will greatly increase the likelihood that the captured BE progression biomarkers will have sufficient positive predictive value to be clinically actionable. Since the MM-TCEB procedure will be less expensive and less burdensome than endoscopy, it will accelerate the pace with which targeted biomarker discovery studies can be conducted, decreasing the cost and time that it will take to identify and clinically translate the best biomarker panels. When made widely available, this technology will decrease EAC mortality while curbing costs by making available a population-based means for screening for BE progressors, so that all such patients are identified and treated, while the majority of those with BE who are at low risk will avoid unnecessary endoscopy.

Broadly speaking, the development of OCT-TCE has enabled unsedated, microscopic imaging of the entire esophagus using a swallowable, tethered capsule. Our lab has mass produced micromotor beam scanning tethered capsules (FIG. 6A) and briefcase-sized OCT imaging systems (FIG. 6B), using them to obtain OCT data (FIG. 6C) and 3D images (FIG. 6D) of the esophagus in over 500 unsedated patients. Results show that OCT-TCE is a safe, rapid (5 minute), well tolerated, and accurate method for detecting BE that can be implemented by nurses/technicians in primary care settings with minimal training. As OCT-TCE is a depth-resolved microscopic imaging technology that images the entire esophagus, it can distinguish esophagus from stomach tissue (e.g. by visualization of esophageal submucosal glands and palisading vessels) to differentiate BE from cardiac metaplasia and is exquisitely sensitive to sub-mm regions of BE at the GEJ.

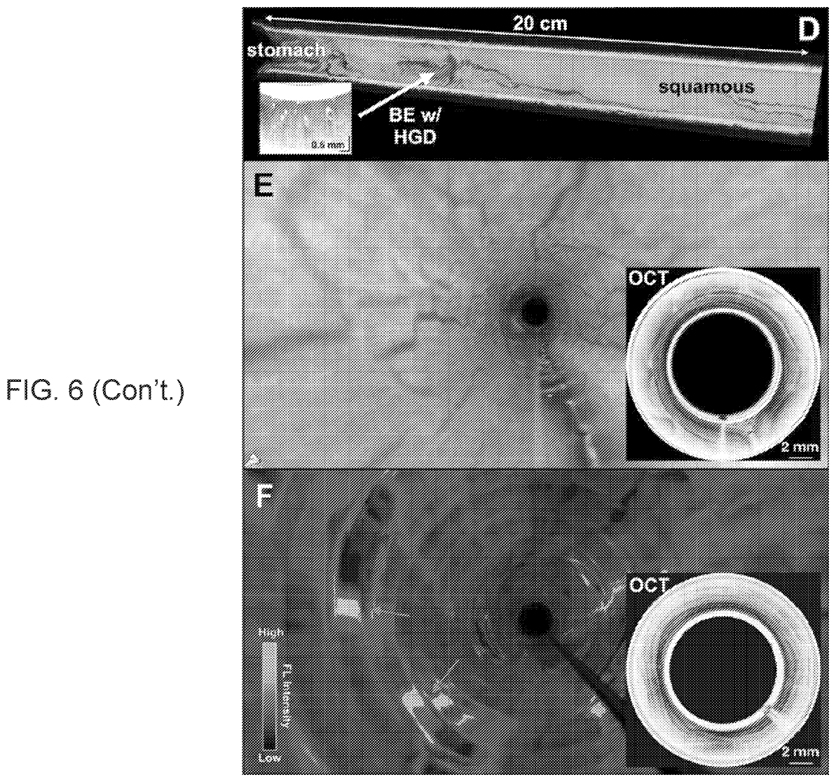

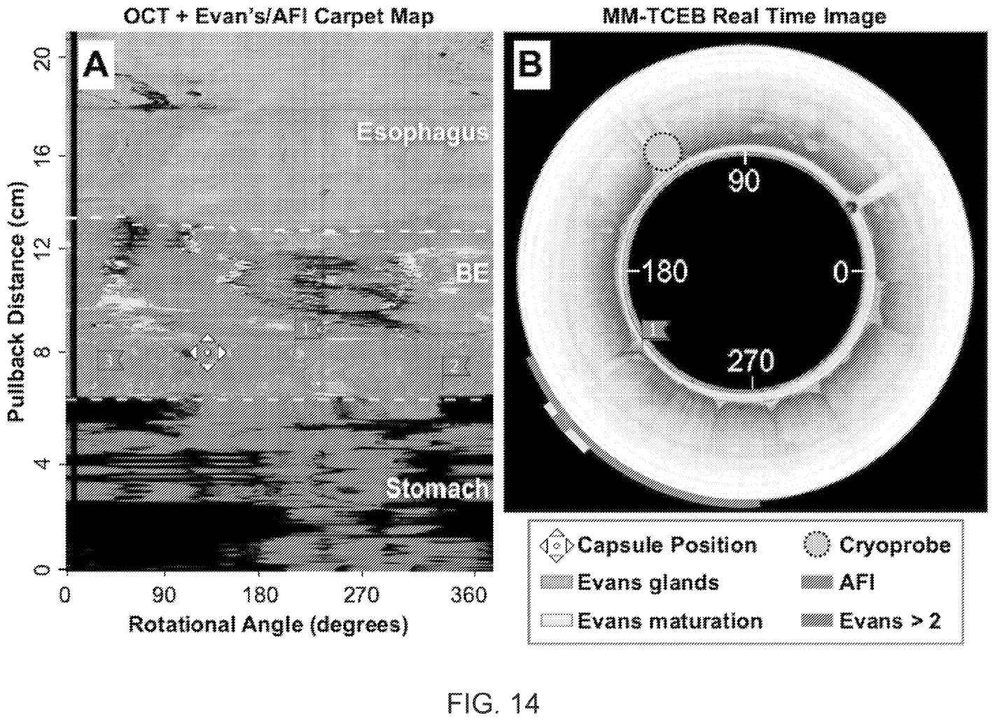

We additionally have pioneered multimodality TCE, demonstrating both white light RGB-OCT and near-infrared fluorescence (NIRF)-OCT tethered capsules that employ DCFs in vivo (FIGS. 6E, 6F). As disclosed herein, multimodality TCE imaging will be extended by incorporating a new two-fiber design that enables AF/R detection without incurring sensitivity losses caused by high DCF fluorescence background at UV/blue AF excitation wavelengths. Unlike prior MM-OCT TCE devices that use single detector schemes, the disclosed imaging system will detect both OCT and AF/R spectra; the latter can be converted into standard 3-color RGB AFI images (FIG. 7; R=green reflectance, G=total autofluorescence, B=green reflectance). In so doing, capsule-based AFI can be used to target esophageal tissue rich in molecular alterations, an approach that has been utilized in seminal endoscopic biomarker discovery studies. OCT, shown to be capable of detecting architectural abnormalities that are indicative of early neoplastic changes, will be combined with AFI in a machine-learning-enabled, real time, guided biopsy user interface (FIG. 14).

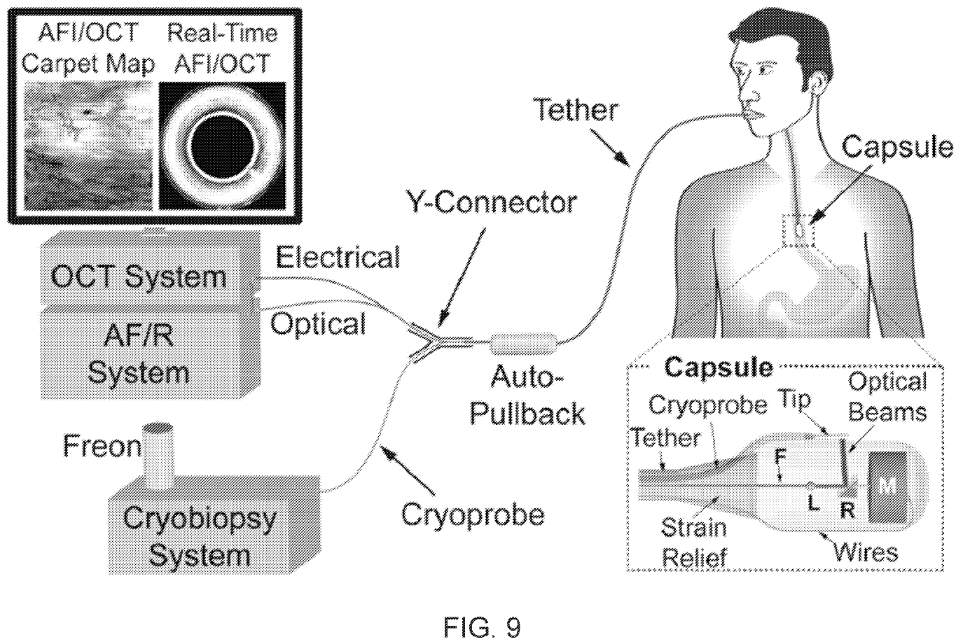

While cryobiopsy is used clinically for lung biopsy, we have improved the technology by developing portable Freon-based cryobiopsy systems (FIG. 8A) and ˜1-mm-diameter, throttle-enabled, OCT-guided cryoprobes that can readily attach to any medical device (FIGS. 8A, 8B). We have successfully utilized this cryobiopsy technology to obtain upper gastrointestinal (GI) biopsies that are superior (e.g. larger, fewer artifacts) to conventional forceps biopsies in over 30 animals and patients in vivo (FIGS. 8C, 10H). The MM-TCEB tether will have a unique flexible/torqueable design that will allow the cryoprobe to be easily directed to the tissue of interest under real time OCT guidance. In some embodiments the torqueable tether may include commercially available braided sheaths which may have metal windings embedded in another material (e.g. as shown in FIGS. 10A, 10B; from Duke Extrusion, Braided Stock Tubing, PTFE Liner w/Pebax™ 55D Jacket, 0.147″OD/0.130″ID (11.3Fr), 48 in long). This first-of-a-kind image-targeted capsule biopsy system will not only collect tissue for BE biomarkers but will also enable less invasive biopsy of cancer and other conditions of the upper GI tract (including ulcers, celiac disease, eosinophilic esophagitis, etc.). Since biopsy depth can be tailored by altering the time that the cryoprobe's cold tip is on the tissue, the device can additionally acquire deeper biopsies for assessing GI strictures, mural inflammation, and tumor staging.

In various embodiments, clinical studies will be conducted to demonstrate that the diagnostic accuracy of biomarkers derived from MM-TCEB samples is equivalent to that of biomarkers from AFI-targeted endoscopic biopsies. While these results are important for the clinical translation of MM-TCEB, this rich and unique data set of co-registered OCT and AF/R spectra contains information beyond that of intensity-based OCT/AFI that can be used to further improve targeting accuracy. One reason we chose to combine OCT and AF/R spectroscopy in the capsule is based on prior work which suggests that AF/R techniques can uncover intrinsic chemical/molecular features that should be indicators of BE progression such as nuclear size, porphyrins, hemoglobin content/oxygenation, collagen/elastin, and NAD(P)H (hereafter NADH):FAD (optical redox ratio). The diagnostic potential of some of these AF/R spectroscopy modalities has previously been demonstrated in BE patients undergoing sedated endoscopy but this is the first time that these methods will be incorporated into a swallowable tethered capsule that can be used in unsedated subjects. Also, since OCT and AF/R will be co-registered, quantitation will be superior and less subject to artifact. Indeed, the clinical significance and ultimately the accuracy of this device can be increased by placing the AF/R data in the context of OCT microanatomy, calibrating AF/R spectra by OCT-measured capsule-lumen distance, and correcting AF attenuation using tissue reflectance (absorption).

Newer depth dependent OCT algorithms advanced here have the potential to provide additional information relevant to BE progression. The OCT correlation of the derivative bandwidth (COD BW), which is used to ascertain nuclear size (aneuploidy), and OCT-derived group velocity dispersion (GVD), which is used to assess microscopic molecular changes, have only been recently described by our team and demonstrated ex vivo. Thus, we will use the imaging and biopsy dataset to investigate the relationships between these new image-derived parameters and tissue-derived BE progression biomarkers. Moreover, we will incorporate features extracted from OCT and AF/R spectroscopy, combined with patient demographic information, in a deep learning pipeline that will increase targeting precision by combining the complementary data streams. Deep learning innovations will include: (i) the use of 2D and 3D convolutional neural networks (CNN) that will account for the correlations between adjacent images and/or other data in stacks, (ii) the use of multi-modal data and state-of-the-art data fusion to merge intermediate features extracted from various deep learning models, and (iii) optimization of custom network architectures using evolutionary techniques. In addition to improving the precision of image-based targeting beyond that of standard OCT/AFI, this data analysis research will be an important step towards determining whether tethered capsule imaging can be used to identify BE progressors without taking tissue from the body.

### Development of Multimodality TCE Biopsy (MM-TCEB) Technology for Targeted Sampling of BE Tissue

### MM-TCEB Overview

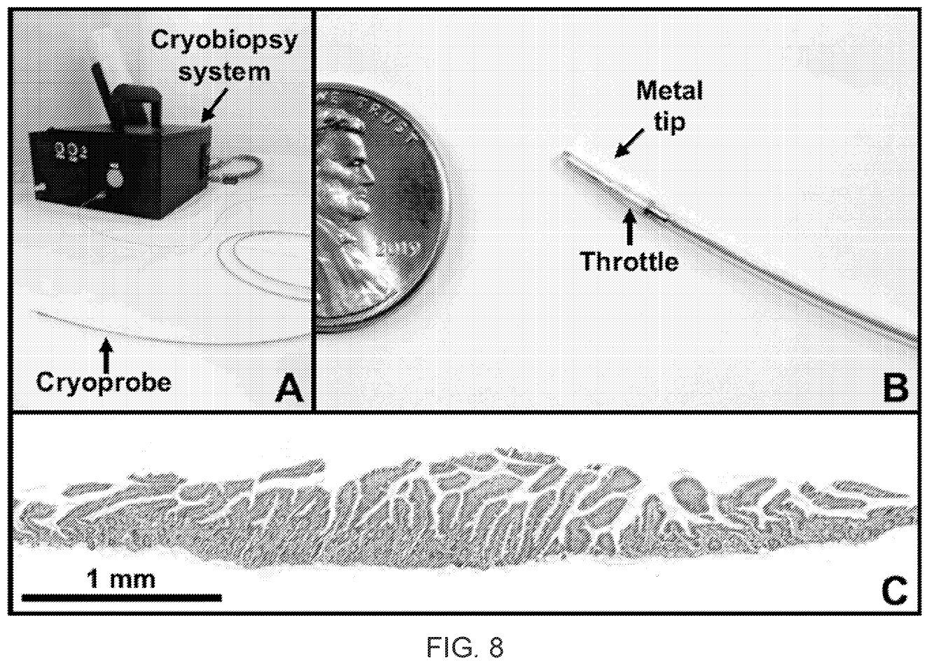

Embodiments of the MM-TCEB system will include an OCT system and control software, an AF/R spectroscopy imaging system, a cryobiopsy system, the MM-TCEB tethered capsule device, and a cryoprobe (FIG. 9). Optical fibers for the OCT and AF/R spectroscopy imaging systems, electrical motor-control wires, and the cryoprobe will be combined through the input ports of a Y-connector to reside within a flexible, torqueable tether. The tether will be clamped within an automatic pullback device that can draw it and the capsule back through the esophagus at a constant rate. The capsule will be attached to the tether via an interposed strain relief, configured so that the cryoprobe can be inserted through the tether/strain relief and around the capsule's body (FIG. 9, right lower inset). Inside the capsule, the fibers (F) will be terminated by a lens (L) such as a ball lens or a GRIN lens; converging light from the lens will illuminate a reflector (R) that is mounted to the shaft of an integrated micromotor (M). When present, the cryoprobe will obscure the optical beams so that its tip is visible in the OCT cross-sectional image (FIG. 10C).

### MM-TCEB Targeted Biopsy Procedure

After the patient swallows the MM-TCEB capsule, the operator will let it descend into the stomach, confirmed by visualization of characteristic OCT images “pit and crypt” features. Once in the stomach, the automated pullback device will pull the tether back at a constant velocity (˜2 mm/s), storing OCT and AF/R data and displaying OCT/AFI cross-sectional images in real time. The 3D OCT and AF/R dataset for the first pullback will be processed to generate an OCT dysplasia (Evan's Criteria: glandular atypia and poor surface maturation) and AFI carpet map view (e.g., FIG. 14). The user will click on the screen, applying flags on the carpet map denoting locations to biopsy (regions that contain magenta-purple AFI and/or OCT areas with an Evan's Dysplasia Score >2). After the biopsy sites are selected, the capsule will descend to the stomach again and a second pullback will commence. During this second pullback, the capsule's location will be updated on the carpet map in real time. When the capsule's position is at the same esophageal level as a carpet map flag (targeted biopsy site), the operator will pause pullback and insert the cryoprobe into the tether until its tip is seen in the OCT image (FIG. 10C) and will rotate the tether until the cryoprobe's tip is over the targeted location (FIGS. 10D-10G). Once imaging confirms cryoprobe-target contact, Freon will be injected into the cryoprobe, cooling its tip for 5-10 seconds at approximately −30° C., affixing tissue to the probe. The cryoprobe with the attached frozen tissue will be withdrawn through the tether and the tissue collected for histology, IHC, and cytometry. This process will be repeated for all targeted biopsies.

### MM-TCEB Tethered Capsule Device

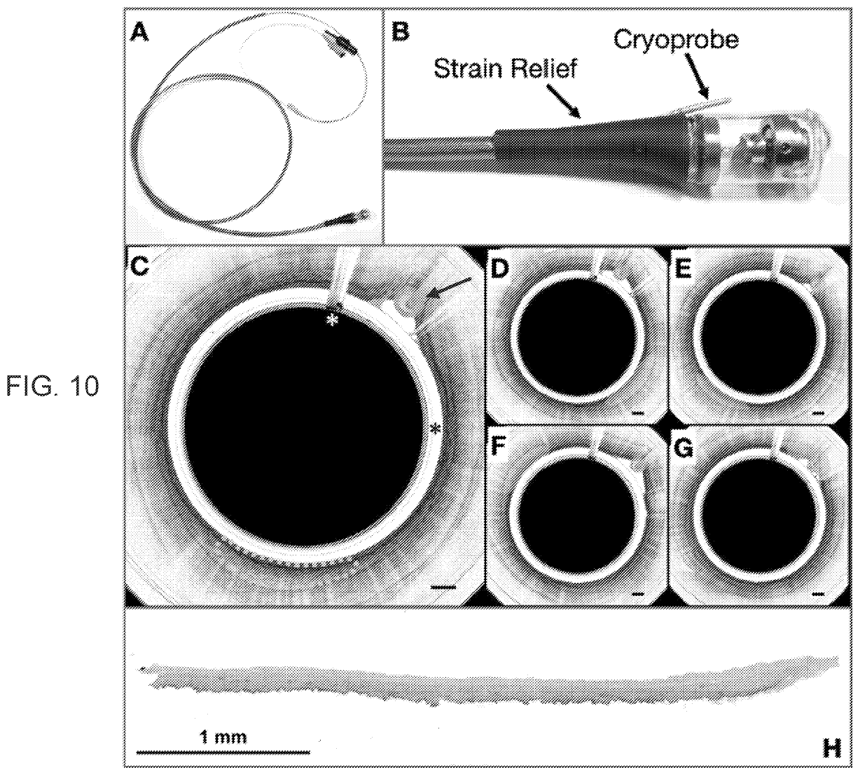

MM-TCEB device: In various embodiments, the proposed capsule shell will be comprised of optically clear PMMA and will have dimensions that have been effectively utilized with high swallowing rates in multicenter and primary care studies. The cryoprobe provision will be custom molded and embedded in a silicone strain relief The prototype's existing 1.2-mm-diameter cryoprobe can only traverse a 20° bend over the 11-mm-diameter capsule, requiring a longer strain relief than necessary (FIG. 10B). To reduce the capsule and strain reliefs' lengths, in certain embodiments the capsule's diameter may be decreased to 8 mm; the cryoprobe's diameter may be changed to 0.8 mm and the length of the metal tip of the cryoprobe may be changed to 5 mm, and the tether may be offset from the capsule's central axis (see diagram in FIG. 11A). In some embodiments, the tether may include braided sheath windings to maximize torquability and flexibility, achieving a tether outer diameter of ˜2 mm. The cryoprobe will be isolated within its own channel inside the tether to avoid contamination of the fibers/wires with the cryoprobe or extracted tissue (see FIG. 4, lower panel, and FIG. 11B).

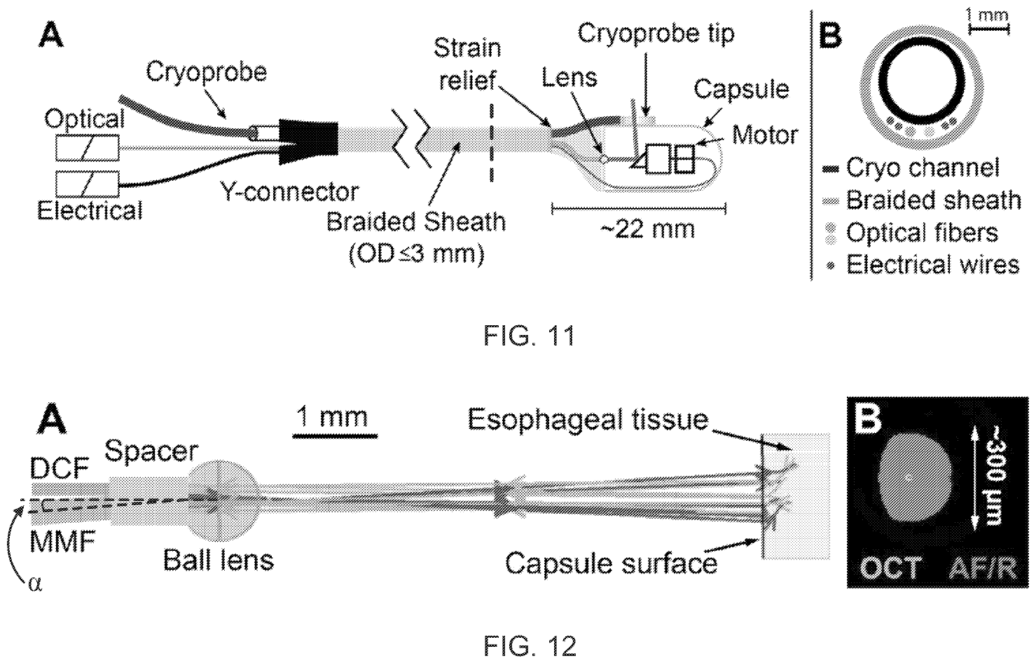

Capsule optics: The MM-TCEB capsule's optics (FIG. 12A) will be similar to that of previously validated, motor-based OCT and NIRF-OCT capsules, with the exception that two fibers, a DCF and an MMF, will be utilized to prevent background fiber autofluorescence (FIG. 12A). This two-fiber configuration has been used to implement RGB-OCT TCE (FIG. 6E). Single mode OCT light will be transmitted through the core of the DCF, and white light and AF remission will be collected through the DCF's inner cladding (diameter-125 μm; NA=0.22). White light illumination (400-700 nm) and AF excitation light (ex: 375 and 450 nm) will be transmitted by an adjacent low-OH MMF (core diameter-200 μm; NA=0.22) (FIG. 12A). Both fibers will be attached to a ball lens that will place the 35 μm (FWHM) OCT focus at the capsule's outer surface (FIG. 12A), centered in a ˜300 m diameter AF/R illumination/collection spot (FIG. 12B); in certain embodiments, the capsule optics may include a GRIN lens instead of, or in addition to, other lenses such as a ball lens. In some embodiments, the ball lens may have an extended spacer coupled thereto, where the DCF and MMF fibers may be coupled at one end of the spacer and the ball lens may be coupled at the other end of the spacer. The MMF in certain embodiments may be attached at an angle α relative to the optical axis (where the optical axis may be defined by the ball lens and/or spacer, if present) to adjust the focal location of the AF/R illumination light on the tissue so that it overlaps the OCT and FL collection foci (FIG. 12B). In various embodiments the angle α may range from greater than 0° to as much as 30°, and in particular embodiments (e.g. FIG. 2A) may be 12°. Monte Carlo modeling (Zemax non-sequential mode, Mie+fluorescence) using optical properties of the esophagus showed that this configuration will enable collection of AF/R at a depth of up to ˜300 μm. OCT and AF/R light will be continuously scanned and collected from the entire circumference of the esophagus at a rate of 40 Hz.

Optical calibration and testing: In various embodiments, calibration tables will be developed for each capsule to correct for wavelength-dependent variations in throughput, AF/R collection, and distance from tissue. A point spread function phantom and z-scanning techniques will be used to evaluate the axial/lateral resolutions of OCT. Reflectance calibration tables will be obtained by illuminating a phantom with the same optical properties as that of BE tissue. Fluorescence will be calibrated using 1 mm capillary tubes of AF430; if the capsule is capable of detecting a 30 nM solution, the signal-to-noise ratio (SNR) will be deemed sufficient (450 nm excitation; see sensitivity analysis below). Defocus response will be determined by translating the phantom and AF430 capillary tubes away from the capsule while using the spectrometer to record intensity information. When conducting MM-TCEB imaging, the distance between the capsule and the tissue surface will be determined by OCT in real time; this distance will be input into the defocus response calibration table to normalize AF/R spectral data. Established methods will be used to correct AF based on reflectance imaging.

Mechanical testing: Tether torquability/flexibility will be tested using the Instron 68SC-5; 2527-303 and in swine studies ex vivo and in vivo. Based on preclinical preliminary studies, metrics of success will include the device being capable of traversing 40 cm through the esophagus, withstanding torques of 2 N·m. For swallowability, tether stiffness with the cryoprobe inserted should be <5×10⁻⁵ N/m², which is like that of other tethers that have been utilized in prior esophageal TCE clinical studies. In preliminary studies, we found that effective biopsy guidance requires a 1800 torque response of 2-seconds.

### MM-TCEB Imaging Systems

OCT system: The MM-TCEB OCT imaging system, based on an Axsun SS-OCT engine, has been fabricated by our lab over 30 times and used for OCT-TCE studies in more than 500 patients. To develop some of the advanced imaging biomarker algorithms that rely on spectral interference data, we will modify the OCT system to acquire raw OCT spectral fringe data, as opposed to the standard configuration that returns JPEG intensity images. OCT will run at an A-line rate of 100 kHz, a B-scan (2500 A-lines) rate of 40 Hz, with an SNR of ˜110 dB, and an axial resolution of ˜10 μm (air). The OCT system will contain motor-control electronics and a computer that will save and output processed data to the user interface in real time.

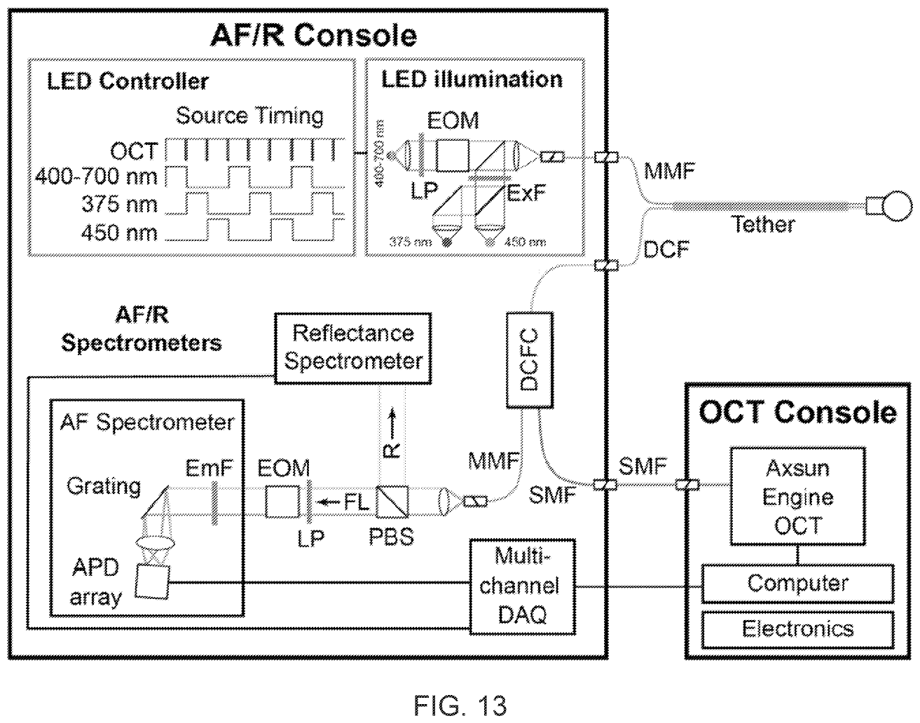

AF/R Imaging System: FIG. 13 shows an embodiment of an MM-TCEB OCT imaging system. Diffuse reflection illumination will be generated using a broadband phosphor-based LED (400-700 nm, white light). AF will be excited by separate narrow band LEDs (e.g. 375 nm and 450 nm LEDs) with center wavelengths that adequately separate NADH and FAD AF, respectively, while staying in the wavelength range of clinical white light endoscopes. Each LED will be alternated every three OCT A-lines (33.3 kHz) (see timing diagram in upper left panel of FIG. 13). In various embodiments, the reflectance LED will be phosphor based as discussed above; due to the phosphorescence lifetime/decay time of the phosphor, such an LED is not completely off (i.e. dark) when modulated above 10 kHz. Thus, in certain embodiments a broadband electro-optical modulator (EOM, Inrad Optics, PKC21-SG15) will be synchronized with this LED's waveform to act as an optical switch and ensure an extinction ratio of >1,500:1, keeping fluorescence crosstalk below 5%. OCT and AF/R light returning from the DCF will be separated into an SMF and MMF using a DCF coupler (DCFC, FIG. 13). A polarized beam splitter will separate reflectance and AF MMF light. An 80 kHz commercial spectrometer with a 2048 element silicon array will detect reflectance light (Wasatch Photonics, CS400-700/300-130-U3). AF will be filtered and then separated by a blazed grating into 16 spectral bins (450-650 nm, 12.5 nm/bin), illuminating an avalanche photodiode array (Hamamatsu S15249). Another EOM (Inrad Optics, PKC21-SG25) will be cycled on and off during reflectance imaging to protect the APDs from damage and avoid detector saturation.

Sensitivity Analysis: The SNR needed for high quality AF/R was simulated for AFI, AF_{ex:375nm}/AF_{ex:450nm} (NADH/FAD), and R₄₀₀₋₇₀₀ₙₘ. Each model used wavelength ranges of interest and respective AF/R spectra from normal and diseased esophageal tissue previously reported in the literature. The spectra were binned into the proposed 16 (AF) or 2048 (R) detector pixels. An AFI model was used to evaluate the SNR required to distinguish high-grade dysplasia from NDBE and normal tissue (p<0.05). For AF_{ex:375nm}/AF_{ex:450nm}, an optical redox (NADH/FAD) model was used to determine the SNR that will separate NADH from FAD AF in esophageal tissue and generate a redox ratio (+/−10%). For R₄₀₀₋₇₀₀ₙₘ, a diffuse reflectance model was used to discriminate squamous, NDBE, and HGD/IMC using the average attenuation spectra of the different tissue types (p<0.05). To approximate the anticipated SNR for each of the three models, we used Zemax coupling values, and then estimated realistic downstream losses, including fiber losses (10%), DCFC coupling (50%), free space optics losses (30%), and grating inefficiencies (15%). We then accounted for detector specific binning, electronic bandwidth, dark current, digitization noise, shot noise, and quantum efficiencies to determine threshold SNRs. Our findings showed that the SNRs required to distinguish these features were 11.4 dB, 10.2/14.3 dB, and 10.4 dB for AFI, AF_{ex:375nm}/AF_{ex:450nm}, and R₄₀₀₋₇₀₀ₙₘ, respectively. Using the Zemax coupling simulations with the proposed two-fiber optical configuration, AF/R system optical loss estimations, information from detector datasheets, and assuming 10 mW AF excitation (375 or 450 nm), and 20 mW broadband reflectance illumination, the models showed that the minimum average per-channel SNRs for the proposed AF/R system should be 16.4 dB, 10.3/16.5 dB, and 19.9 dB for AFI, AF_{ex:375nm}/AF_{ex:450nm}, and R₄₀₀₋₇₀₀ₙₘ, respectively. All modeled SNRs for the proposed AF/R system exceed the requirements for spectral feature discrimination.

### MM-TCEB Cryoprobe and System

Embodiments of the cryobiopsy system may include a slot for a 2-lb coolant (e.g. R410A Freon) tank, a set of two solenoid valves (Redhat, ¼″ Cryogenic Solenoid Valve, ⅛ in Orifice Dia., 120V AC) and a timer electronic circuit. The electronic circuit will generate a waveform to precisely control the duration over which the two solenoid valves are open, pumping coolant into the cryobiopsy probe. The cryoprobe will comprise a 1.2-m-long PTFE sheath, a distal metallic tip, and a hand-held controller to activate cryobiopsy acquisition and manipulate the cryoprobe during biopsy capture. The existing 1.2-mm-diameter cryoprobe will be redesigned to have a smaller rigid length (5.0 mm) and outer diameter (0.8 mm) by adjusting its sheath's cross-section and the solenoid control waveform to enable better coolant expansion and probe cooling.

### MM-TCEB Software

After the first MM-TCEB pullback, two-dimensional maps representing Evan's criteria (glands) and poor surface maturation (bright surface reflectance), and AFI and enface OCT will be overlaid and displayed on the screen (FIG. 14A). BE and Evan's criteria will be classified using CNNs. Upon double clicking on this carpet map, a flag corresponding to an intent to biopsy location (e.g., Evan's criteria score >2 and/or magenta AFI) will be registered and displayed (FIG. 14A). This process will be repeated for all targeted biopsy regions. During the second pullback, the cross-sectional MM-TCE OCT image and associated AFI and Evan's features will be dynamically shown on the screen (FIG. 14B). The capsule's current location will be updated in real time on the carpet map using the tether's position, determined by the automatic pullback device's encoder, and adjusted/confirmed by cross correlation of the first and second pullbacks' OCT/AFI images. Previously placed flags will be updated on the real time cross-sectional image in a similar manner. When the level of the capsule is the same as that of a flagged region, the operator will hold the capsule still at the incisors, insert the cryoprobe, and visualize the cryoprobe's tip on the cross-sectional image (FIGS. 10C-10G, 14B). The tip will then be rotated by torquing the tether (FIGS. 10C-10G). A cryobiopsy will be taken once the tip is overlaid on the cross-sectional image's flag. The capsule will then be pulled back to the next flagged location until all targeted biopsies have been acquired.

### System Validation

OCT SNR and axial resolution will be validated using standard techniques. For AF/R, the system's SNR will be tested by coupling LED light with known optical powers and spectra into the detectors. Standard methods will be used to validate the accuracy of the CNNs for BE and Evan's criteria classifications.

### Biopsy Targeting Accuracy Testing in Swine In Vivo

The ability of the MM-TCEB technology to accurately target biopsies will be tested preclinically in normal swine (n=10; 50:50 male/female; 50-80 kg). After swine are anesthetized, they will undergo endoscopy; ˜2-mm-diameter cautery marks will be generated in the esophagus to serve as cryobiopsy targets (FIGS. 10C-10G). The endoscope will be removed and the MM-TCEB capsule will be inserted into the esophagus. Targeted cryobiopsies of the cautery sites will be obtained per the methods described above. Targeting accuracy will be determined by visualization of cauterized tissue in the H&E histology slides. With 5 cautery marks/swine, this study will test 2-mm targeting accuracy of 90%±10% (95% CI).

### Reproducibility, AFI, and Biopsy Testing in Humans In Vivo

Clinical tethered capsules and cryoprobes will be developed using a design control process, validated preclinically, and made in a class-10000 clean room for fabricating clinical-grade GI devices. The capacity of the MM-TCEB device to acquire clinical standard AFI images and operate as intended will be tested in BE patients undergoing evaluation for dysplasia (n=20) in vivo. This study will also evaluate the test-retest reproducibility of carpet map acquisition. Approximately one week prior to MM-TCEB, patients will undergo research sedated endoscopy with an Olympus GIF-FQ260Z upper endoscope and the CV-260SL video processor that have been used in prior AFI clinical biomarker studies. A movie comprising AFI images will be recorded during controlled pullback of the scope through the esophagus. No biopsies will be taken. After recovering from the endoscopy imaging procedure, the same study subjects will swallow the MM-TCEB capsule without sedation. During the first pullback, OCT/AFI carpet maps will be generated, and targeted biopsy sites flagged (3-4 abnormal and one normal), as described above. To test reproducibility, a second pullback will be repeated to create second AFI carpet maps. During a third pullback, the capsule/cryoprobe will be used to obtain cryobiopsies at the flagged sites. Cryobiopsies will be considered adequate if their width is >2 mm, the depth is >200 μm, and histopathologic quality is deemed satisfactory by expert pathologist analysis of H&E slides. Targeting feasibility will be assessed by determination of the capacity to biopsy the flagged sites. Carpet maps will be generated from the Olympus AFI endoscope data and warped to the MM-TCEB AFI carpet maps using landmarks (vessels, gastroesophageal junction, common OCT/AFI features) present in both datasets. Test-retest carpet map pairs will be registered as above, augmented by recorded pullback tether positions. Correspondences between test/retest MM-TCEB AFI and AFI endoscopy carpet maps will be quantified by Mander's overlap and Pearson's correlation coefficients. 20 patients will test statistical significance (p<0.05) for a correlation r≥0.6 with 80% power.

### Potential Difficulties, Limitations, and Alternative Approaches

Cost/complexity: The MM-TCEB device developed here is not intended to be a final screening solution but instead a validation of the concept of tethered capsule biopsy targeting for BE progressors. Once we demonstrate feasibility, costs/complexity can be decreased by using economical OCT architectures and by measuring AF/R at a few discrete wavelengths.

Automated biopsy: This initial foray into capsule-based, image-guided biopsy is manual and may require training/skill that could be a barrier to wide dissemination. Should the manual approach be effective, a next step could be to automate the biopsy process using real-time image analysis and user-assisted robotics.

Narrow-Band Imaging (NBI): If we find that OCT and AFI insufficiently target biopsies that contain key BE progression biomarkers, other techniques that have been shown to increase accuracy for detecting dysplasia such as narrow-band-imaging (NBI) can be derived from our data and incorporated into this targeted biopsy platform.

Sensitivity: Should we encounter light levels that are lower than expected, we can increase illumination power either by using higher power LEDs or by switching to other sources such as lasers. We can also slow the scan speed or add extra multimode fibers to the MM-TCEB device for increased remittance collection.

Tissue capture reliability: In rare cases (<5%), the esophagus does not contact the capsule, potentially rendering cryobiopsy ineffective. This issue is resolved clinically by stopping the capsule and waiting for peristalsis to re-engage. Applying suction through the cryobiopsy channel would also be effective in ensuring tissue contact.

Comparing MM-TCE AFI to endoscopic AFI: It may be challenging to register and warp endoscopic AFI to MM-TCE AFI and derive high correlations between the two. This potential issue can be mitigated by data reduction such as the use of block carpet maps that integrate over the entire circumference to generate one AFI value per frame or by comparing % AFI-positive areas.

### Determination of the Capacity of MM-TCEB to Acquire Targeted Tissue Samples Containing BE Progression Biomarkers In Vivo

Here, we will validate MM-TCEB technology by demonstrating that the biomarkers obtained from tissue captured by this device represent dysplasia status, a surrogate for BE progression risk. This clinical study is based on the validation phase of di Pietro et al. (di Pietro M, et al. Gut. 2015; 64(1):49-56, which is incorporated herein by reference in its entirety), with the main difference being that the AFI-targeted biopsy will be obtained using the less invasive MM-TCEB technology in unsedated subjects as opposed to AFI-targeted endoscopic biopsy in patients under conscious sedation. This will also acquire a rich dataset of OCT and AF/R spectra, AFI, and registered BE tissue samples and biomarkers (p53, cyclin A, aneuploidy, dysplasia status), enabling the development of new algorithms for improving biomarker targeting.

Patients: Patients scheduled for elective upper endoscopy for evaluation or treatment of dysplastic BE or EAC at MGH will be enrolled in this study. We will enroll 125 patients since our goal is to conduct the MM-TCEB procedure in 100 and the swallowing rate for these devices is ˜80%. Patient characteristics (e.g., age, sex, diet, body habitus, medications, and GI and GERD histories) will be recorded.

Protocol summary: Patients will undergo MM-TCEB 1-2 weeks prior to or after their scheduled standard of care sedated high-resolution endoscopy (HRE). During endoscopy, biopsies of visible lesions and 4-quadrant random biopsies will be taken as per the Seattle protocol. MM-TCEB will be conducted unsedated with the acquisition of OCT/AFI-targeted cryobiopsies, according to the procedure disclosed above (see MM-TCEB targeted biopsy procedure). On average, there will be 3-4 cryobiopsies taken from OCT/AFI-targeted regions (OCT/AFI positive) and one from an OCT/AFI-negative region. The duration of the MM-TCEB procedure will be approximately 30 minutes. Cryobiopsies will be thawed and bisected along their longitudinal axis (the cryoprobe procures long tissue hemicylinders). Half will be sent for cytometry and the other half will be Formalin-fixed and paraffin-embedded (PPFE). Questionnaires about subject experience with the MM-TCEB procedure will also be administered immediately after the study.

Sterilization and reuse: The MM-TCEB device will be designed for up to 5 uses. After the tethered capsule is removed from the subject, it will be cleaned and disinfected for reuse in accordance with the standard procedure for the high-level disinfection of GI endoscopes, including the Endozime InstruSponge™ for flexible instruments.

Histopathology: H&E slides from all MM-TCEB biopsies and standard of care endoscopic biopsies will be read by three blinded pathologists with experience in Barrett's diagnosis (Drs. G. Tearney, M. Pitman, M. Mino-Kenudson). Dysplasia will be independently diagnosed by each pathologist according to the Montgomery/Vienna classification schemes. A consensus diagnosis will be achieved through a review of discordant cases.

Biomarker analysis: Biomarkers previously established to accurately predict dysplasia and BE progression, p53, cyclin A, and aneuploidy, will be measured as described in di Pietro et al. (referenced above). Briefly, p53 IHC slides will be generated and scored by the 3 blinded pathologists as positive for cases where staining is strong (overexpression) or lost (underexpression). Cyclin A will be deemed to be positive if 1% of surface cells exhibit staining. Flow cytometry analysis to evaluate aneuploidy will be conducted using known procedures.

Statistical analysis: Biomarker positivity will be compared to histopathologic assessment of dysplasia status for all biopsies (cryobiopsies and endoscopic biopsies). Diagnoses will be on a per biopsy and per patient basis.

Sample size rationale: Assuming a sensitivity for a three-biomarker panel (p53, cyclin A, aneuploidy) of 95% and a specificity of 90%, 25 patients with HGD/EAC will result in 95% confidence intervals of +8.5% and +11%, respectively. If 25% of patients have HGD/EGD and the MM-TCEB swallowing rate is 80%, we will need to enroll 125 patients in this study. Enrollment will accrue over a period of 3 years.

Sex and other relevant biological variables: For the data obtained according to the work disclosed herein, we will conduct subgroup analyses and multivariable analyses to determine if the results are different as a function of sex, age, race, BMI, GERD symptoms/chronicity, BE segment length, or other relevant variables.

### Potential Difficulties, Limitations, and Alternative Approaches

AFI false positivity near the gastric folds: Some studies show that the AFI false positive rate is higher near the gastroesophageal junction (GEJ) due to elevated inflammation commonly found at this anatomic site. If co-existent OCT imaging cannot mitigate AFI false positives, we can avoid biopsying AFI-positive areas within 1 cm of the GEJ or limit patient enrollment to those with longer BE segments (Prague criteria C>2 or C<2 & M>4), at the expense of lower enrollment rates.

Dysplasia yield: If the yield of HGD/EAC biopsies is low despite our image-guided sampling approach, we will focus our enrollment to patients with long segment BE and/or patients with a history of dysplasia.

Image cytometry: We will use image cytometry to evaluate aneuploidy if transient tissue freezing during cryobiopsy affects flow cytometry results.

Alternative biomarkers: In this study, we propose to utilize certain exemplary BE progression biomarkers because they have been extensively validated in the literature, and have been shown to be predictive of neoplastic status and BE progression. Should new biomarkers become validated that provide additional information on BE progression, we will include them as time and resources permit.

### Development and Validation of MM-TCE Imaging Biomarkers for BE Progression

We will use advanced image analysis and deep learning techniques to mine data obtained as described above and develop MM-TCE imaging biomarkers that reflect corresponding tissue-derived biomarkers. We will test the hypothesis that the analysis of diffuse reflectance spectra and raw OCT interference fringe data will provide an estimate of epithelial aneuploidy in BE. While OCT and reflectance spectroscopy have independently been shown to extract nuclear size information from human tissues, these techniques have not been specifically combined or validated in patients for BE aneuploidy, a strong predictor of progression. We will also test the hypothesis that deep learning applied to OCT, AF/R spectroscopy, and demographic information can provide metrics that correlate to dysplasia grade (NDBE, LGD, HGD, IMC) and IHC/cytometry of BE progression biomarkers. Deep learning is important for the success of this research where: (i) subtle feature variations will be useful for classification of classes that are difficult to discriminate (e.g., small variations in the spectra reflecting metabolic changes), (ii) the spatial relations/variations of the features are directly related to the classification (e.g., micro-structural changes in OCT images), and (iii) the correlations between data from adjacent regions can be exploited. This work will improve the precision of MM-TCEB image-based targeting, resulting in better biopsy enrichment, and increased accuracy of tissue-derived biomarkers for BE progression prediction. Results will also inform on the potential of an imaging capsule for identifying BE progressors without having to remove tissue from the patient.

### Analysis of Diffuse Reflectance Spectra and Raw OCT Interference Fringe Data Will Provide an Estimate of Epithelial Aneuploidy in BE

Light scattering spectroscopy for nuclear size: The BE biomarker aneuploidy is defined as the existence of distinct populations of nuclei with chromatin content (nuclear size) distributions deviating from the standard diploid (G1) peak profile. Light scattering spectroscopy (LSS) estimates chromatin content through measurement of nuclear size, ascertained via analysis of reflectance spectra remitted from tissue. The intensity of the LSS spectrum varies in wavelength in an oscillatory manner. The frequency/depth of these oscillations depend on the size and number density of the scatterers (cell nuclei). LSS has been successfully used to estimate dysplasia grade in BE in excised specimens and in living human patients. In our device, the LSS spectrum will be extracted from the reflectance spectra by subtracting the diffuse component based on a theoretical model. Variations in frequency and depth of the LSS spectrum will be analyzed with model based light scattering theory to determine cell nuclei size distributions.

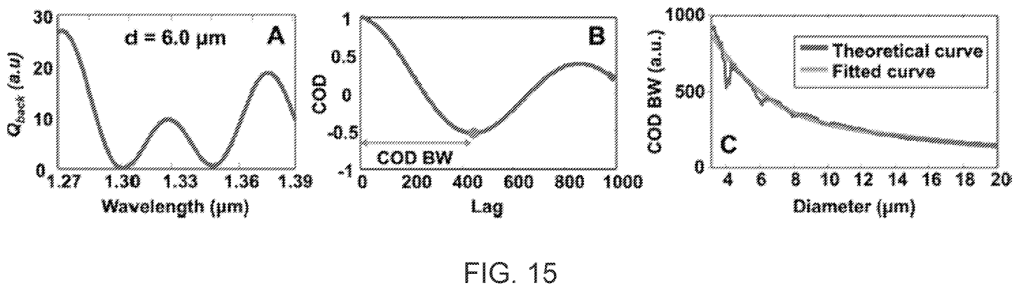

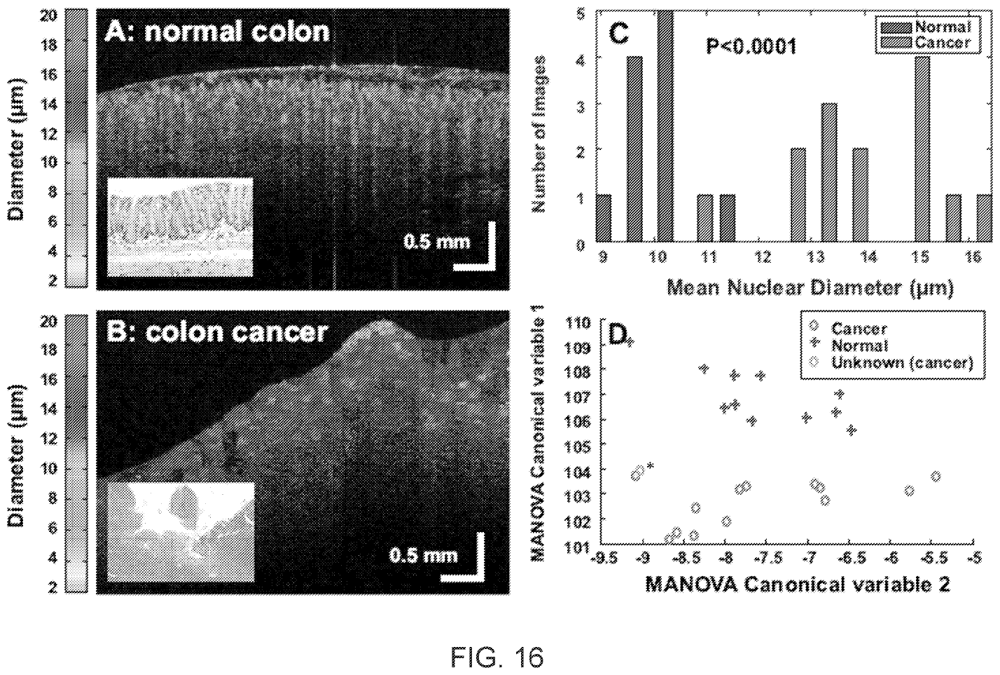

OCT measurements of nuclear size: Like reflectance spectroscopy, the spectra of the back-reflected light, extracted from the OCT interferometric fringe pattern, also exhibit size-dependent fluctuations. Members of our team used this fact and Mie theory to create a new spectral metric for OCT, the correlation of the derivative bandwidth (COD BW), for high-resolution, depth-dependent scatterer size estimation (Kassinopoulos et al., Biomed Opt Express. 2017; 8(3):1598-606, which is incorporated herein by reference in its entirety). The COD is the autocorrelation of the first derivative of the depth-resolved OCT spectrum and the COD BW is the lag position of the first minimum of the COD (FIG. 15B). Once computed, the COD BW can be converted to scatterer size using a Mie theory derived relationship. The feasibility/accuracy of this method for estimating scatterer size was studied using OCT images from microsphere phantoms and human normal/colonic adenocarcinoma tissues (FIG. 16). Increased scatter size diameters measured by COD BW were consistent with expected nuclear changes that occur in cancer (FIG. 16C) and the accuracy of COD BW for classification of normal vs. cancerous samples was high (96%; FIG. 16D).

Exploiting the synergy between visible reflectance LSS and OCT-based COD BW measurements: Since the wavelength ranges of LSS and OCT are different (400-700 nm vs. 1235-1385 nm) and OCT provides depth-resolved data, the information provided by the two techniques is complementary. Spectral variations induced by large scatters have a higher frequency in the visible range and may be challenging to model in the presence of noise and background diffuse light. The spectral fluctuations produced by these same scatterers may be more apparent in the longer OCT wavelength range. Conversely, OCT is not as suitable for detecting small scattering sizes (<2 μm), such as those induced by cellular organelles and chromatin variations, which are more evident in the visible spectrum. Furthermore, LSS is performed by extracting a small percentage of single-scattered light from a background of multiply scattered photons, using approximate models. Due to unknown variations in tissue scattering properties, this modeling can be challenging, leading to difficulty fitting spectral oscillations to Mie theory, resulting in less accurate values. Since OCT detects single-scattered photons, we will extend the Mie theory model of OCT to shorter wavelengths to correct for uncertainties in visible wavelength LSS measurements.

In vitro validation: As there are no well-established in vitro models for EAC, these novel imaging-based aneuploidy markers will initially be validated using normal melanocyte spheroids and melanoma spheroids that are known to exhibit aneuploidy, comprised of de-differentiated (RPMI7951) human melanoma cells (n=20 per type). Each spheroid will be imaged by MM-TCE; LSS and OCT COD BW nuclear size distributions will then be extracted from the data. Assuming a difference in LSS/OCT COD BW metrics between diploid and aneuploid cell histogram means of 50% and a standard deviation of 50% of the mean (as shown in Kassinopoulos et al. referenced above), a total of 20 spheroids in each group will be required to show a one-sided statistical significance with a power of 0.8 and an a of 0.05. Spheroids will also undergo flow cytometry to obtain cell DNA content histograms that will be compared to LSS/OCT COD BW nuclear size distributions via the two-sample Kolmogorov-Smirnov statistic.

Ex vivo validation: LSS and OCT COD BW aneuploidy metrics will be validated ex vivo by imaging tissue biopsy samples from Barrett's patients (n=20 NDBE and n=20 HGD/IMC) with the MM-TCE device. Samples will be imaged fresh with the luminal side up in iso-osmolar phosphate buffered saline. Biopsies will then undergo flow cytometry; analyses and sample size calculations follow those described above.

In vivo validation: AF/R and OCT images obtained from MM-TCEB targeted biopsy sites in vivo will be analyzed to extract LSS and OCT COD BW nuclear size distributions. For a training set (roughly 10%), metrics obtained in vivo will be combined to create an imaging-based aneuploidy index. In the validation set, aneuploidy by OCT COD BW, LSS, and their combination will be compared to gold standard cytometry diagnosis of aneuploidy from corresponding biopsies obtained as disclosed above. Assuming a sensitivity/specificity of 90%, an intra-patient correlation of 25%, 400 biopsies with 35% prevalence of aneuploidy 25% HGD+10% NDBE/LGD), will enable 95% confidence intervals of ±5%. LSS and OCT COD BW nuclear size distributions will also be compared to cytometry cell DNA content histograms using the two-sample Kolmogorov-Smirnov statistic.

Deep Learning Applied to OCT, AF/R Spectroscopy, and Demographic Information Can Provide Metrics that Correlate to Dysplasia Grade (NDBE, LGD, HGD, IMC) and IHC/Cytometry of BE Progression Biomarkers

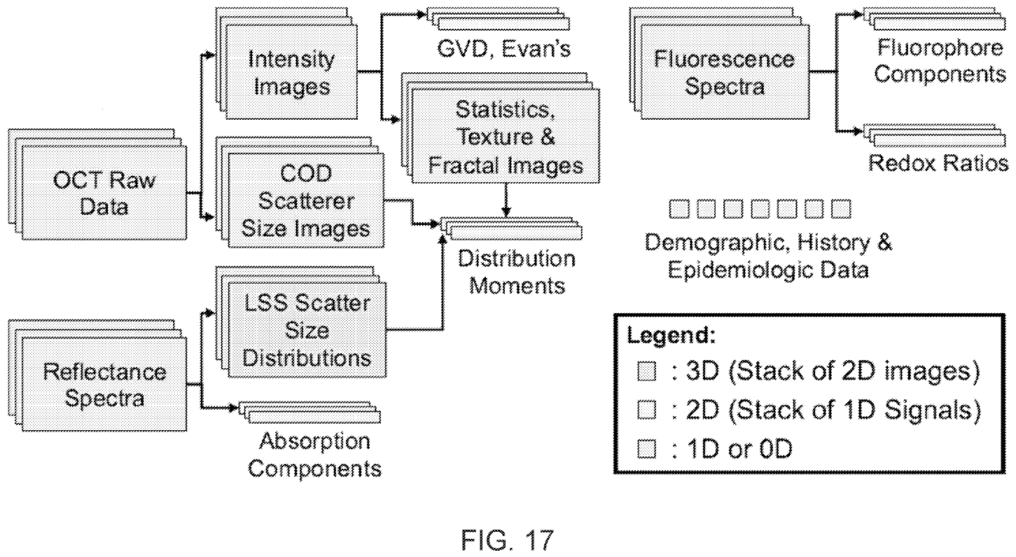

Feature extraction: To fully leverage the richness of the MM-TCEB data, we will follow the strategy of extracting additional information from the raw data, followed by multimodal deep learning using CNNs and autoencoders. Information that will be extracted from the MM-TCEB data is summarized in FIG. 17; metrics/methods that have not been discussed in detail previously are described below.

Estimation of metabolism from the autofluorescence spectra: Since extracellular components such as elastin and collagen also contribute to AF at UV/blue excitation wavelengths, we will initially unmix the fluorescent spectra to obtain the contributions of NADH and FAD using a least squares approach with the addition of “unknown components” extracted using principal component analysis or spectral band ratios. We will also investigate the use of spectral phasor analysis that is advantageous over conventional linear unmixing, as it is rapid and does not require apriori knowledge of basis emission spectra.

Group velocity dispersion (GVD) estimate from OCT speckle patterns: GVD is a measure of the variation of the refractive index of tissue as a function of the wavelength of light. Methods have been developed for measuring GVD from OCT speckle in vivo without the need of distinct reflectors (Photiou et al. Biomed Opt Express. 2017; 8(5):2528-35, which is incorporated herein by reference in its entirety). This technique is based on the estimation of the degradation of the point spread function (PSF) that is extracted from the dispersion-induced variation of the speckle pattern as a function of depth. The potential relevance of this method for diagnosing neoplasia was shown by GVD classification of OCT images of human normal and cancerous colon tissue ex vivo with 93% sensitivity and 100% specificity (96% overall accuracy).

OCT intensity statistics: Additional features will be extracted from the OCT images, selected due to their capability to quantify significant sub-resolution variations that affect the intensity of the backscattered signal and image texture. These metrics can be grouped into four categories: intensity first-order statistics (FOS), gray level co-occurrence matrix (GLCM) features, grey-tone difference matrix (GTDM) features and fractal dimension statistics. Using these intensity features, COD BW, and GVD, our group has shown that a neural network-based approach can separate NDBE from dysplasia with an overall accuracy of 89%.

Data integrity and preprocessing: All data collected will first undergo quality assessment by the investigational team. Before using the datasets for training the algorithms, preprocessing steps such as calibration, capsule-to-lumen distance correction, attenuation correction, normalization, and/or filtering will be used to assure data consistency. The effect of missing or inconsistent data will be assessed and imputation strategies for dealing with gaps in the data will be implemented.

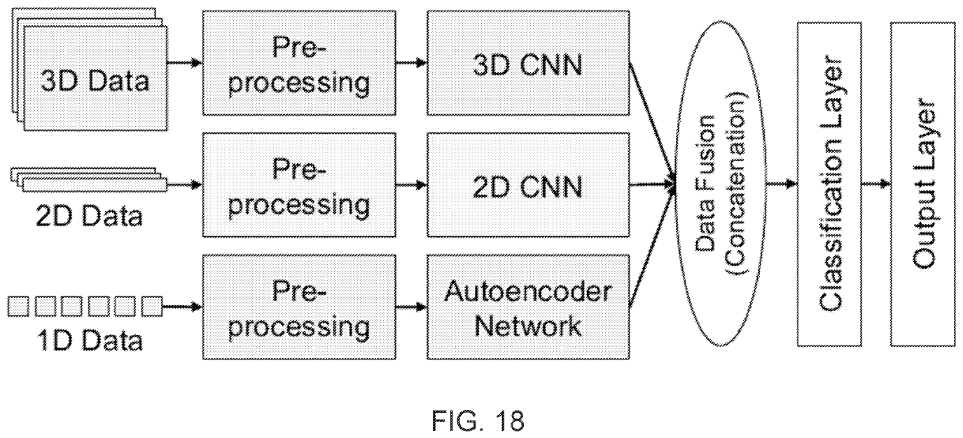

Deep learning architecture: The proposed custom deep learning architecture is shown in FIG. 18. The training of the data from each modality will be initially executed separately using different deep learning models. Specifically, for imaging or spatially resolved spectral data, a 3D convolutional neural network architecture, consisting of convolutional, pooling, and full connection layer, will be used to train and extract features. Similarly, a 2D CNN will be used for 2D data. For the remaining data types (demographics, clinical history, OCT-determined BE length, etc.), back propagation training will be performed using stacked autoencoder neural networks, with multiple hidden layers, to acquire deep data characteristics and extract features. After training is completed, an intermediate data fusion strategy will be used to combine the intermediate features extracted from all data modalities using a deep model. Concatenation-based data fusion inherits the merits from both a raw feature level and decision-level integration to further improve prediction accuracy. A concatenating layer will be used to merge the intermediate features extracted from all deep learning models, before feeding them through the second level of a fully connected layer. For the classification layer, several alternative methods will be used such as k-nearest neighbor, random forests, support vector machines, and/or another CNN. To obtain high classification accuracy, the architecture, parameters, and initialization for each of the deep learning models will be determined using an evolutionary algorithm, leveraging its global optimization characteristics. We will utilize the SHapley Additive exPlanation (SHAP) framework to interpret the model and to understand the relative contributions of the different features in the final classifications.

Comparison of outputs to BE progression biomarkers gold standards: The outputs of the classification layer will consist of categorical ordinal outputs of tissue dysplasia stage (NHBE, LGD, HGD, IMC) and binary outputs for aberrant p53 expression, cyclin A overexpression, and aneuploidy, effectively performing a 7-class classification. The sensitivity, specificity, and AUC of each output will be determined using corresponding histology and/or IHC/cytometry from the collected biopsies. Validation of the models will be performed by LOOCV or by randomly splitting the data into training and test sets, starting at a 10:90 ratio and increasing training until the models are stable. Analyses will be performed on a per biopsy basis and a per patient basis. The latter leverages OCT and AF/R spectroscopy of the entire BE segment, greatly increasing the training set volume and potentially picking up a neoplastic field effect. The additional ˜16 biopsies/patient available from the standard endoscopic procedure following MM-TCEB will be used to refine the per patient dysplasia diagnosis.

Data size considerations: The planned data set of 400-500 colocalized 3D OCT intensity and interferometric data, AF/R spectra, cryobiopsies, and biomarkers (H&E, p53, Cyclin A, aneuploidy) from over 100 BE patients will be more than adequate to train and validate the deep learning models (>1000 2D sets per class). Assuming that biopsies and corresponding OCT images are 3 mm in length/width and the pullback speed is 2 mm/s, the dataset created for each biopsy will consist of 60 OCT images and 600 independent AF/R spectra. The OCT interferometric data will be processed to extract scatterer size distributions (6 2D statistical moments of scatterer per OCT image) and the OCT intensity will be further analyzed to create statistical and fractal distributions (˜20 2D features×6 statistical moments per OCT image) and GVD and Evan's criteria data. AF/R images will be used to obtain spectrally unmixed component images and AF redox ratio data. On a per-patient basis, the data size increases by two orders of magnitude, assuming a 4-cm-long BE segment.

### Computing System

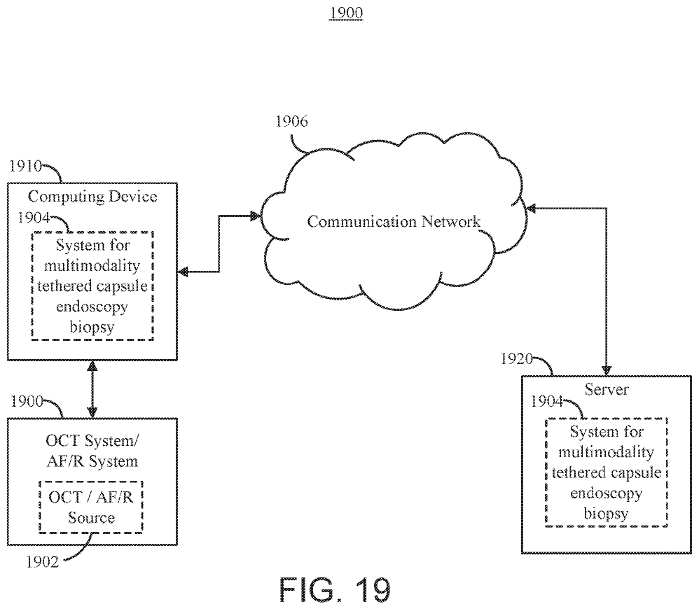

Turning to FIG. 19, an example 1900 of a system (e.g. a data collection and processing system) for multimodality tethered capsule endoscopy biopsy is shown in accordance with some embodiments of the disclosed subject matter. As shown in FIG. 19, a computing device 1910 can receive data (e.g. OCT data, reflectance data, and/or autofluorescence data) from an OCT/AF/R system 1900. In some embodiments, computing device 1910 can execute at least a portion of a system for multimodality tethered capsule endoscopy biopsy 1904 to identify an area of interest in a sample based on data obtained from OCT/AF/R system 1900. Additionally or alternatively, in some embodiments, computing device 1910 can communicate information about the data received from OCT/AF/R system 1900 to a server 1920 over a communication network 1906, which can execute at least a portion of system for multimodality tethered capsule endoscopy biopsy 1904 to identify an area of interest in the sample based on the data. In some such embodiments, server 1920 can return information to computing device 1910 (and/or any other suitable computing device) indicative of an output of system for multimodality tethered capsule endoscopy biopsy 1904, such as the area of interest. This information may be transmitted and/or presented to a user (e.g. a researcher, an operator, a clinician, etc.) and/or may be stored (e.g. as part of a research database or a medical record associated with a subject).

In some embodiments, computing device 1910 and/or server 1920 can be any suitable computing device or combination of devices, such as a desktop computer, a laptop computer, a smartphone, a tablet computer, a wearable computer, a server computer, a virtual machine being executed by a physical computing device, etc. As described herein, system for multimodality tethered capsule endoscopy biopsy 1904 can present information about the data and/or the area of interest to a user (e.g., researcher and/or physician).

In some embodiments, OCT/AF/R system 1900 may include an OCT/AF/R source 1902, which can be any source suitable for optical interferometry such as OCT and/or for autofluorescence and diffuse reflectance spectroscopy. In other embodiments, OCT/AF/R source 1902 can be local to computing device 1910. For example, OCT/AF/R source 1902 may be incorporated with computing device 1910 (e.g., computing device 1910 can be configured as part of a device for multimodality tethered capsule endoscopy biopsy). As another example, OCT/AF/R source 1902 may be connected to computing device 1910 by a cable, a direct wireless link, etc. Additionally or alternatively, in some embodiments, OCT/AF/R source 1902 can be located locally and/or remotely from computing device 1910, and can communicate information to computing device 1910 (and/or server 1920) via a communication network (e.g., communication network 1906).

In some embodiments, communication network 1906 can be any suitable communication network or combination of communication networks. For example, communication network 1906 can include a Wi-Fi network (which can include one or more wireless routers, one or more switches, etc.), a peer-to-peer network (e.g., a Bluetooth network), a cellular network (e.g., a 4G network, a 5G network, etc., complying with any suitable standard, such as CDMA, GSM, LTE, LTE Advanced, WiMAX, etc.), a wired network, etc. In some embodiments, communication network 1906 can be a local area network, a wide area network, a public network (e.g., the Internet), a private or semi-private network (e.g., a corporate or university intranet), any other suitable type of network, or any suitable combination of networks. Communications links shown in FIG. 19 can each be any suitable communications link or combination of communications links, such as wired links, fiber optic links, Wi-Fi links, Bluetooth links, cellular links, etc.

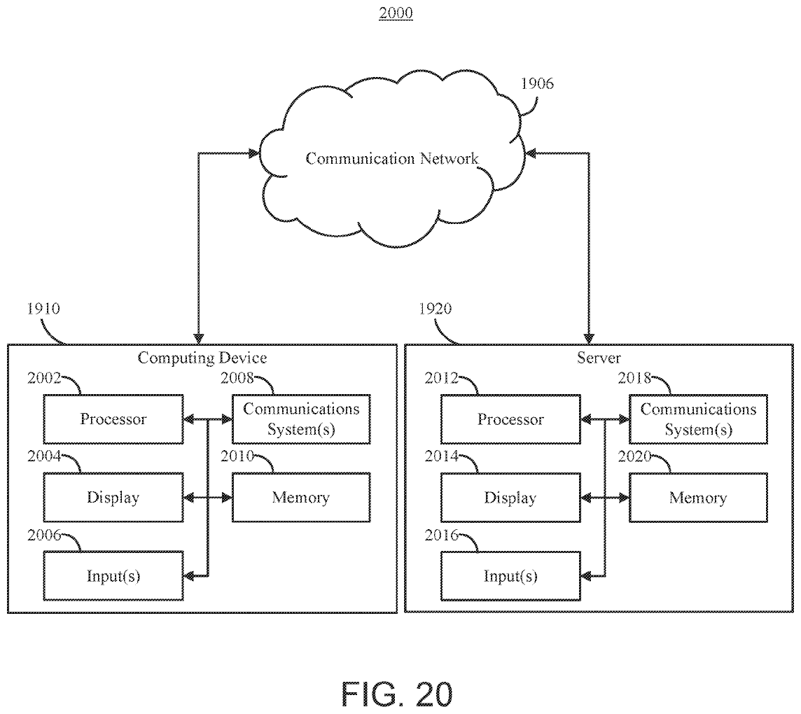

FIG. 20 shows an example 2000 of hardware that can be used to implement computing device 1910 and server 1920 in accordance with some embodiments of the disclosed subject matter. As shown in FIG. 20, in some embodiments, computing device 1910 can include a processor 2002, a display 2004, one or more inputs 2006, one or more communication systems 2008, and/or memory 2010. In some embodiments, processor 2002 can be any suitable hardware processor or combination of processors, such as a central processing unit, a graphics processing unit, etc. In some embodiments, display 2004 can include any suitable display devices, such as a computer monitor, a touchscreen, a television, etc. In some embodiments, inputs 2006 can include any suitable input devices and/or sensors that can be used to receive user input, such as a keyboard, a mouse, a touchscreen, a microphone, etc.

In some embodiments, communications systems 2008 can include any suitable hardware, firmware, and/or software for communicating information over communication network 1906 and/or any other suitable communication networks. For example, communications systems 2008 can include one or more transceivers, one or more communication chips and/or chip sets, etc. In a more particular example, communications systems 2008 can include hardware, firmware and/or software that can be used to establish a Wi-Fi connection, a Bluetooth connection, a cellular connection, an Ethernet connection, etc.

In some embodiments, memory 2010 can include any suitable storage device or devices that can be used to store instructions, values, etc., that can be used, for example, by processor 2002 to present content using display 2004, to communicate with server 1920 via communications system(s) 2008, etc. Memory 2010 can include any suitable volatile memory, non-volatile memory, storage, or any suitable combination thereof. For example, memory 2010 can include RAM, ROM, EEPROM, one or more flash drives, one or more hard disks, one or more solid state drives, one or more optical drives, etc. In some embodiments, memory 2010 can have encoded thereon a computer program for controlling operation of computing device 1910. In such embodiments, processor 2002 can execute at least a portion of the computer program to present content (e.g., images, user interfaces, graphics, tables, etc.), receive content from server 1920, transmit information to server 1920, etc.

In some embodiments, server 1920 can include a processor 2012, a display 2014, one or more inputs 2016, one or more communications systems 2018, and/or memory 2020. In some embodiments, processor 2012 can be any suitable hardware processor or combination of processors, such as a central processing unit, a graphics processing unit, etc. In some embodiments, display 2014 can include any suitable display devices, such as a computer monitor, a touchscreen, a television, etc. In some embodiments, inputs 2016 can include any suitable input devices and/or sensors that can be used to receive user input, such as a keyboard, a mouse, a touchscreen, a microphone, etc.

In some embodiments, communications systems 2018 can include any suitable hardware, firmware, and/or software for communicating information over communication network 1906 and/or any other suitable communication networks. For example, communications systems 2018 can include one or more transceivers, one or more communication chips and/or chip sets, etc. In a more particular example, communications systems 2018 can include hardware, firmware and/or software that can be used to establish a Wi-Fi connection, a Bluetooth connection, a cellular connection, an Ethernet connection, etc.

In some embodiments, memory 2020 can include any suitable storage device or devices that can be used to store instructions, values, etc., that can be used, for example, by processor 2012 to present content using display 2014, to communicate with one or more computing devices 1910, etc. Memory 2020 can include any suitable volatile memory, non-volatile memory, storage, or any suitable combination thereof. For example, memory 2020 can include RAM, ROM, EEPROM, one or more flash drives, one or more hard disks, one or more solid state drives, one or more optical drives, etc. In some embodiments, memory 2020 can have encoded thereon a server program for controlling operation of server 1920. In such embodiments, processor 2012 can execute at least a portion of the server program to transmit information and/or content (e.g., results of a tissue identification and/or classification, a user interface, etc.) to one or more computing devices 1910, receive information and/or content from one or more computing devices 1910, receive instructions from one or more devices (e.g., a personal computer, a laptop computer, a tablet computer, a smartphone, etc.), etc.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (such as hard disks, floppy disks, etc.), optical media (such as compact discs, digital video discs, Blu-ray discs, etc.), semiconductor media (such as RAM, Flash memory, electrically programmable read only memory (EPROM), electrically erasable programmable read only memory (EEPROM), etc.), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

In some embodiments, the optical signals are detected by photodiodes. It should be recognized that any opto-electronic conversion device including but not limited to photo detectors, photodiodes, line-scan and two-dimensional cameras, and photodiode arrays can be used to perform this detection function.

It should be noted that, as used herein, the term mechanism can encompass hardware, software, firmware, or any suitable combination thereof.

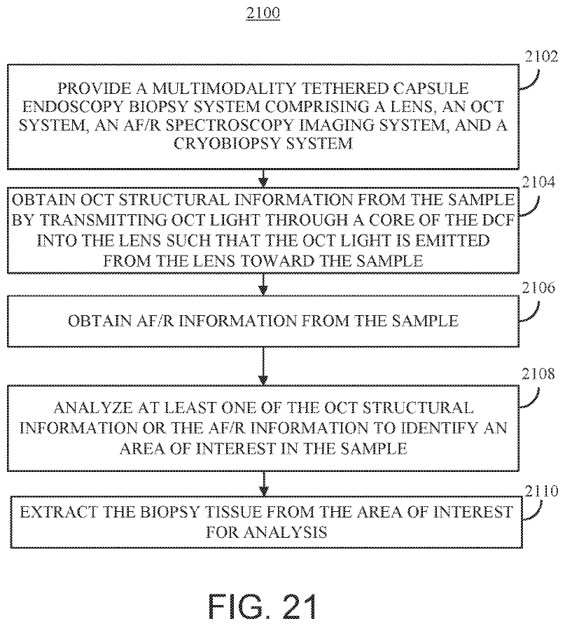

FIG. 21 shows an example 2100 of a process for multimodality tethered capsule endoscopy biopsy in accordance with some embodiments of the disclosed subject matter. As shown in FIG. 21, at 2102, process 2100 can provide a multimodality tethered capsule endoscopy biopsy system comprising a lens, an optical coherence tomography (OCT) system, an autofluorescence and diffuse reflectance (AF/R) spectroscopy imaging system, and a cryobiopsy system. The lens may include a double clad fiber (DCF) and a multimode fiber (MMF) coupled thereto, the optical coherence tomography (OCT) system may include an OCT light source, the AF/R spectroscopy imaging system may include an AF/R light source, and the cryobiopsy system may include a cryobiopsy probe configured to be placed in a field of view of the OCT system to obtain a biopsy tissue from a sample. At 2104, process 2100 can obtain OCT structural information from the sample by transmitting OCT light from the OCT light source through a core of the DCF into the lens such that the OCT light is emitted from the lens toward the sample, where the OCT structural information may be obtained using the OCT system. At 2106, process 2100 can obtain AF/R information from the sample, which may include transmitting AF/R light from the AF/R light source through the MMPF into the lens such that the AF/R light is emitted from the lens toward the sample, and collecting the AF/R information remitted from the sample via an inner cladding of the DCF. AF/R information may be obtained from the sample using the AF/R spectroscopy system. At 2108, process 2100 can analyze at least one of the OCT structural information or the AF/R information to identify an area of interest in the sample. Finally, at 2110, process 2100 can extract the biopsy tissue from the area of interest for analysis, where the tissue may be extracted using the cryobiopsy probe.

It should be understood that the above described steps of the process of FIG. 21 can be executed or performed in any order or sequence not limited to the order and sequence shown and described in the figures. Also, some of the above steps of the processes of FIG. 21 can be executed or performed substantially simultaneously where appropriate or in parallel to reduce latency and processing times.

Thus, while the invention has been described above in connection with particular embodiments and examples, the invention is not necessarily so limited, and that numerous other embodiments, examples, uses, modifications and departures from the embodiments, examples and uses are intended to be encompassed by the claims attached hereto.

What is claimed is:

1. An imaging and biopsy device, comprising:
a tethered capsule that is configured to be swallowed;
a first optical fiber transmitting an electromagnetic radiation that at least partially impacts an anatomical structure; and
a biopsy apparatus configured to collect tissue from the anatomical structure,
the electromagnetic radiation at least partially or temporarily impacting the biopsy apparatus, and
at least a portion of the first optical fiber and the biopsy apparatus being associated with the tethered capsule.

2. The device of claim 1, wherein the first optical fiber comprises at least one of a single mode fiber (SMF) or a double clad fiber (DCF).

3. The device of claim 1, wherein the tether is configured to be torqueable.

4. The device of claim 1, wherein the biopsy apparatus comprises a cryobiopsy apparatus.

5. The device of claim 1, wherein the electromagnetic radiation is optically coupled to an optical coherence tomography (OCT) system.

6. The device of claim 5, further comprising a detector system optically coupled to the first optical fiber that generates an OCT image,
wherein the biopsy apparatus is at least partially visible in the OCT image.

7. An imaging and biopsy device, comprising:
a tethered capsule that is configured to be swallowed;
a first optical fiber transmitting a first electromagnetic radiation and a second electromagnetic radiation that at least partially impact an anatomical structure;
a second optical fiber receiving a third electromagnetic radiation that is emitted from the anatomical structure,
a wavelength of the third electromagnetic radiation being different from a wavelength of the first electromagnetic radiation and a wavelength of the second electromagnetic radiation; and
a biopsy apparatus configured to collect tissue from the anatomical structure,
at least one of the first electromagnetic radiation or the second electromagnetic
radiation at least partially or temporarily impacting the biopsy apparatus, and
at least a portion of the first optical fiber, the second optical fiber, and the biopsy apparatus being associated with the tethered capsule.

8. The device of claim 7, wherein the first optical fiber comprises at least one of a single mode fiber (SMF) or a double clad fiber (DCF), and
wherein the second optical fiber comprises a multimode fiber (MMF).

9. The device of claim 7, wherein at least one of the first electromagnetic radiation or the second electromagnetic radiation excites fluorescence in the anatomical structure to generate the third electromagnetic radiation.

10. The device of claim 7, wherein the tether is configured to be torqueable.

11. The device of claim 7, wherein the biopsy apparatus comprises a cryobiopsy apparatus.

12. The device of claim 7, wherein the first electromagnetic radiation is optically coupled to an optical coherence tomography (OCT) system.

13. The device of claim 12, further comprising a detector system optically coupled to the first optical fiber that generates an OCT image,
wherein the biopsy apparatus is at least partially visible in the OCT image.

14. The device of claim 7, further comprising a fourth electromagnetic radiation and a fifth electromagnetic radiation remitted from the anatomical structure,
wherein the fourth electromagnetic radiation is transmitted to a reflectance spectroscopy system, and
wherein the fifth electromagnetic radiation is transmitted to a fluorescence spectroscopy system.

15. A multimodality tethered capsule endoscopy biopsy system, comprising:
a lens comprising a double clad fiber (DCF) and a multimode fiber (MMF) coupled thereto;
an optical coherence tomography (OCT) system comprising an OCT light source,
the OCT light source configured to transmit OCT light through a core of the DCF into the lens such that the OCT light is emitted from the lens toward a sample;
an autofluorescence and diffuse reflectance (AF/R) spectroscopy imaging system comprising an AF/R light source,
the AF/R light source configured to transmit AF/R light through the MMF into the lens such that the AF/R light is emitted from the lens toward the sample, and
the AF/R spectroscopy imaging system configured to collect the AF/R light remitted from the sample via an inner cladding of the DCF; and
a cryobiopsy system comprising a cryobiopsy probe configured to be placed in a field of view of the OCT system to obtain tissue from the sample.

16. The system of claim 15, further comprising a sleeve into which the DCF and the MMF are disposed.

17. The system of claim 16, wherein the sleeve comprises a channel disposed therein,
wherein the cryobiopsy probe is disposed within the channel to be placed in the field of view of the OCT system.

18. The system of claim 17, wherein the sleeve comprises a strain relief at a distal end thereof,
wherein a distal end of the channel is coupled to the strain relief, and
wherein the strain relief comprises an opening in a lateral portion thereof through which the cryobiopsy probe extends into the field of view of the OCT system.

19. The system of claim 18, wherein the lens comprises a ball lens.

20. The system of claim 19, further comprising an extended spacer having a long axis,
wherein the ball lens is coupled to a distal end of the spacer, and
wherein the DCF and the MMF are coupled to a proximal end of the spacer.

21. The system of claim 20, wherein the DCF is coupled to the distal end of the spacer in an orientation parallel to the long axis of the spacer, and
wherein the MMF is coupled to the distal end of the spacer at an angle relative to the long axis of the spacer.

22. The system of claim 21, further comprising a reflector located distal to the ball lens and disposed at an angle relative to the long axis of the spacer to direct light from the ball lens toward the sample.

23. The system of claim 22, further comprising a motor coupled to the reflector,
wherein the motor is configured to rotate the reflector about the long axis of the ball lens.

24. The system of claim 23, further comprising a capsule coupled to the distal end of the sleeve via the strain relief,
wherein the reflector, the ball lens, the spacer, and the motor are disposed within the capsule.

25. The system of claim 24, wherein the sleeve comprises at least one of a torque coil or a braided sheath.

26. The system of claim 15, wherein the AF/R light source comprises a broad spectrum light source configured to provide light for diffuse reflectance imaging and at least one narrow band light source configured to stimulate autofluorescence in the sample.

27. The system of claim 26, wherein the broad spectrum light source comprises an electro-optic modulator (EOM) configured to intermittently block or allow transmission of output from the broad spectrum light source to the MMF.

28. The system of claim 26, wherein the at least one narrow band light source comprises an LED light source configured to be switched on or off.

29. The system of claim 28, wherein the LED light source comprises a plurality of LED light sources configured to emit light at 375 nm and 450 nm and configured to be switched at a rate of 100 kHz.

30. The system of claim 15, wherein the AF/R light collected by the AF/R spectroscopy imaging system comprises AF/R spectra, and
wherein clinical standard color autofluorescence imaging (AFI) images are generated based on the AF/R spectra.

31. The system of claim 15, wherein the lens comprises a GRIN lens.

32. The system of claim 15, wherein the cryobiopsy system further comprises a coolant, and
wherein the cryobiopsy system is configured to inject the coolant into the cryobiopsy probe.

33. A method for multimodality tethered capsule endoscopy biopsy, comprising:
providing a multimodality tethered capsule endoscopy biopsy system comprising a lens, an optical coherence tomography (OCT) system, an autofluorescence and diffuse reflectance (AF/R) spectroscopy imaging system, and a cryobiopsy system,
the lens comprising a double clad fiber (DCF) and a multimode fiber (MMF) coupled thereto,
the optical coherence tomography (OCT) system comprising an OCT light source,
the AF/R spectroscopy imaging system comprising an AF/R light source, and
the cryobiopsy system comprising a cryobiopsy probe configured to be placed in a field of view of the OCT system to obtain a biopsy tissue from a sample;
obtaining, using the OCT system, OCT structural information from the sample by transmitting OCT light from the OCT light source through a core of the DCF into the lens such that the OCT light is emitted from the lens toward the sample;
obtaining, using the AF/R spectroscopy system, AF/R information from the sample, comprising:
transmitting AF/R light from the AF/R light source through the MMF into the lens such that the AF/R light is emitted from the lens toward the sample, and
collecting the AF/R information remitted from the sample via an inner cladding of the DCF;
analyzing at least one of the OCT structural information or the AF/R information to identify an area of interest in the sample; and
extracting, using the cryobiopsy probe, the biopsy tissue from the area of interest for analysis.

34. The method of claim 33, wherein obtaining OCT structural information from the sample further comprises:
identifying, based on the OCT structural information, the cryobiopsy probe within the field of view of the OCT system.

35. The method of claim 34, wherein extracting the biopsy tissue from the area of interest further comprises:
guiding the cryobiopsy probe to the area of interest based on identifying the cryobiopsy probe within the field of view of the OCT system.

36. The method of claim 35, wherein the cryobiopsy system further comprises a coolant, and
wherein extracting the biopsy tissue from the area of interest further comprises:
injecting the coolant into the cryobiopsy probe.

37. The method of claim 33, wherein transmitting AF/R light further comprises:
emitting light from a broad spectrum light source to provide light for diffuse reflectance imaging, and
emitting light from at least one narrow band light source to stimulate autofluorescence in the sample.

38. The method of claim 37, wherein the broad spectrum light source comprises an electro-optic modulator (EOM), and
wherein emitting light from the broad spectrum light source further comprises:
intermittently blocking or allowing transmission of output from the broad spectrum light source using the EOM.

39. The method of claim 38, wherein the at least one narrow band light source comprises an LED light source, and
wherein emitting light from at least one narrow band light source further comprises:
switching the LED light source on or off.

40. The method of claim 39, wherein the LED light source comprises a plurality of LED light sources comprising a 375 nm LED light source and a 450 nm LED light source, and
wherein switching the LED light source on or off further comprises:
switching the plurality of LED light sources at a rate of 100 kHz.

41. The method of claim 40, wherein transmitting AF/R light further comprises:
alternately transmitting light from the broad spectrum light source, the 375 nm LED light source, and the 450 nm LED light source through the MMF toward the sample.

42. The method of claim 33, wherein the AF/R information comprises AF/R spectra, and
wherein analyzing at least one of the OCT structural information or the AF/R information to identify an area of interest in the sample further comprises:
generating a clinical standard color autofluorescence imaging (AFI) image based on the AF/R spectra, and
analyzing the AFI image to identify the area of interest.

43. The method of claim 42, wherein analyzing the AFI image to identify the area of interest further comprises:
analyzing the AFI image to identify a region of the sample with an increased likelihood of comprising at least one esophageal cancer progression biomarker.

44. The method of claim 43, wherein extracting the biopsy tissue from the area of interest for analysis further comprises:
preparing a histological sample of the biopsy tissue, and
analyzing the histological sample to identify the at least one esophageal cancer progression biomarker.

45. The method of claim 33, wherein analyzing at least one of the OCT structural information or the AF/R information to identify an area of interest in the sample further comprises:
generating, using a deep learning model, at least one metric related to BE dysplasia grade, an esophageal cancer progression biomarker anomaly, or aneuploidy based on at least one of the OCT structural information or the AF/R information, and
identifying the area of interest in the sample based on generating the at least one metric.

46. The method of claim 33, wherein analyzing at least one of the OCT structural information or the AF/R information to identify an area of interest in the sample further comprises:
analyzing the OCT structural information to determine at least one of a correlation of a derivative bandwidth (COD BW) or a group velocity dispersion (GVD), and
identifying the area of interest in the sample based on determining at least one of the COD BW or the GVD.

47. The method of claim 33, wherein analyzing at least one of the OCT structural information or the AF/R information to identify an area of interest in the sample further comprises:
generating an OCT dysplasia and AI carpet map based on the OCT structural information and the AF/R information, and
identifying the area of interest based on the OCT dysplasia and AFT carpet map.

48. The method of claim 47, wherein identifying the area of interest based on the OCT dysplasia and AFI carpet map further comprises:
flagging a location on the OCT dysplasia and AFI carpet map to identify the area of interest, and
wherein extracting the biopsy tissue from the area of interest further comprises:
guiding the cryobiopsy probe to the flagged location, and
extracting the biopsy tissue from the flagged location.

49. The method of claim 33, wherein the MMF is coupled to the lens at an angle α relative to an optical axis of the lens, and
where obtaining AF/R information from the sample further comprises:
transmitting the AF/R light from the AF/R light source through the MMF into the lens at the angle α such that a focal location of the AF/R light overlaps with OCT light and autofluorescence light returned from the sample.

50. The method of claim 33, wherein the multimodality tethered capsule endoscopy biopsy system further comprises a reflector located distal to the lens and a motor coupled to the reflector,
wherein the reflector is disposed at an angle relative to an optical axis of the lens to direct light from the lens toward the sample, and
wherein obtaining OCT structural information from the sample further comprises:
obtaining the OCT structural information from the sample while rotating the reflector, and
wherein obtaining AF/R information from the sample further comprises:
obtaining the AF/R information from the sample while rotating the reflector.
